SEQUENCE LISTING (2 sequences)

SEQ ID NO: 1 - DNA, 22 bp, Artificial Sequence
  Synthetic
gcctcttctcgttcctcatcac

SEQ ID NO: 2 - DNA, 19 bp, Artificial Sequence
  Synthetic
agcagcatcaccgccattg

(19) United States
(12) Patent Application Publication
(10) Pub. No.: US 2024/0307370 A1
(43) Pub. Date: Sep. 19, 2024

(54) SIGMA RECEPTOR LIGANDS FOR TREATING SARS-COV-2 INFECTION

(71) Applicant: University of Florida Research Foundation, Incorporated, Gainesville, FL (US)

(72) Inventors: Christopher R. McCurdy, Gainesville, MA (US); Michael H. Norris, Gainesville, FL (US); David A. Ostrov, Gainesville, FL (US)

(73) Assignee: University of Florida Research Foundation, Incorporated, Gainesville, FL (US)

(21) Appl. No.: 18/275,692

(22) PCT Filed: Feb. 3, 2022
(86) PCT No.: PCT/US2022/015096

§ 371 Date: Aug. 3, 2023

Related U.S. Application Data
(60) Provisional application No. 63/145,807, filed on Feb. 4, 2021.

(51) Int. Cl. A61K 31/4725; A61K 31/138; A61K 31/495; A61K 31/496; A61K 31/55; A61K 38/40; A61K 45/06; A61P 31/14

(52) U.S. Cl. CPC A61K 31/4725; A61K 31/138; A61K 31/495; A61K 31/496; A61K 31/55; A61K 38/40; A61K 45/06; A61P 31/14

(57) ABSTRACT

The invention relates to compounds, compositions, and methods of treating viral infection, especially SARS-CoV-2 infection.

### CROSS-REFERENCE TO RELATED APPLICATIONS

This application claims priority to U.S. Provisional Application No. 63/145,807, filed Feb. 4, 2021, which is incorporated herein by reference in its entirety.

### BACKGROUND

Viruses such as SARS-CoV-2, which is responsible for Coronavirus disease 2019 (COVID-19), continuously evolve as genetic mutations occur during replication of the genome. Multiple variants of SARS-CoV-2 have been documented throughout the COVID-19 pandemic. There is a strong need for prevention and treatment strategies for COVID-19 that are not impacted by SARS-CoV-2 mutations emerging in variants of concern.

### SUMMARY OF THE INVENTION

Compounds having binding affinity for sigma receptors (e.g., sigma-1, sigma-2) are useful to treat and/or prevent viral infection, including SARS-CoV-2 infection. Compounds (e.g., sigma receptor ligands, agonists and antagonists) such as: 4-PPBP (4-phenyl-1-(4-phenylbutyl) piperidine), SA 4503 (cutamesine), ditolylguanidine, dimethyltryptamine, and siramesine are known in the art for sigma receptor binding affinity. AZ 66, CM304, and CM398 also possess binding affinity to sigma receptors. Diphenhydramine, which binds to the Histamine-1 receptor also has off-target sigma-1 receptor binding affinity.

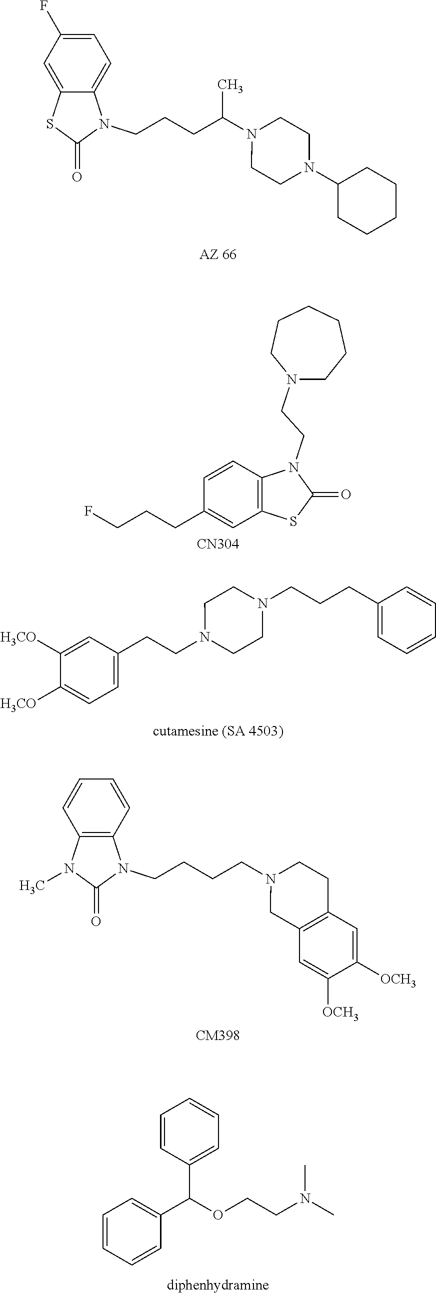

Another object of the present invention is the use of a compound as described herein (e.g., of any formulae herein) in the manufacture of a medicament for use in the treatment of a disorder or disease (e.g., any disorder or disease herein). Another object of the present invention is the use of a compound as described herein (e.g., of any formulae herein) for use in the treatment of a disorder or disease (e.g., any disorder or disease herein). The use in the treatment of a disorder or disease in a subject includes any human, animal, mammal, reptile, bird, and the like.

Thus, in one aspect, provided herein is a method for treating or preventing SARS-CoV-2 infection in a subject, comprising administration to the subject of a compound, or salt thereof, having sigma receptor binding affinity.

In another aspect, provided herein is a method for treating or preventing symptoms of SARS-CoV-2 infection in a subject, comprising administration to the subject of a compound, or salt thereof, having sigma receptor binding affinity.

In another aspect, provided herein is a method for treating or preventing respiratory symptoms of SARS-CoV-2 infection in a subject, comprising administration to the subject of a compound, or salt thereof, having sigma receptor binding affinity.

In another aspect, provided herein is a method for treating or preventing pulmonary dysfunction associated with SARS-CoV-2 infection in a subject, comprising administration to the subject of a compound, or salt thereof, having sigma receptor binding affinity.

In another aspect, provided herein is a method for treating or preventing cardiovascular dysfunction associated with SARS-CoV-2 infection in a subject, comprising administration to the subject of a compound, or salt thereof, having sigma receptor binding affinity.

In another aspect, provided herein is a method for treating or preventing metabolic dysfunction (e.g., pancreatic function) associated with SARS-CoV-2 infection in a subject, comprising administration to the subject of a compound, or salt thereof, having sigma receptor binding affinity.

In another aspect, provided herein is a method for reducing replication of SARS-CoV-2 virus in a subject, comprising administration to the subject of a compound, or salt thereof, having sigma receptor binding affinity.

In another aspect, provided herein is a method for reducing SARS-CoV-2 virus-induced cellular toxicity in a subject, comprising administration to the subject of a compound, or salt thereof, having sigma receptor binding affinity.

In some aspects of any of the above methods, the subject has been identified as having SARS-CoV-2 infection. In some aspects of any of the above methods, the subject has SARS-CoV-2 infection. In some aspects of any of the above methods, the subject has been identified as in need of such treatment. In some aspects of any of the above methods, the subject is in need of such treatment. In some aspects of any of the above methods, the subject is administered a therapeutically effective amount of compound, or salt thereof.

In another aspect, provided herein is a compound that is AZ66, CM304, CM398, or SA4503, or salt thereof, for use in treating or preventing SARS-CoV-2 infection in a subject.

In another aspect, provided herein is a composition comprising a compound that is AZ66. CM304, CM398, or SA4503, or salt thereof, and a pharmaceutically acceptable excipient.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIGS. 1A-1E depict structures of sigma ligands utilized in this and other studies. The structure of sigma ligands are presented with the sigma specific activity, Ki for the respective sigma receptor binding property, and the bioavailability if known. (FIG. 1A) SA4503 (cutamesine) is a selective Sigma-1 piperazine agonist with >15-fold preference for sigma-1 over sigma-2. (FIG. 1B) CM304 is a highly selective benzothiazolone sigma-1 antagonist. (FIG. 1C) CM398 is a highly selective benzimidazolone-based sigma-2 ligand. (FIG. 1D) AZ66 is a mixed sigma-1/sigma-2 selective ligand with an optimized pharmacokinetic profile. (FIG. 1E) PB28 is not utilized in this study but recent work demonstrated its utility in vitro was compromised by its toxicity and is included as a structural comparison to the other compounds.

FIGS. 2A-2H show that highly specific sigma ligands inhibit SARS-CoV-2-induced cytotoxicity in Vero E6 cells. Cytotoxicity measured by LDH release in Vero E6 cells at 72 h in the presence of the indicated drug concentration alone (black bars) or in the presence of the indicated drug after infection with SARS-CoV-2 at an MOI of 0.2 (gray bars) (FIGS. 2A-2D). The 50% cytotoxic concentration (CC₅₀) of the drug alone (black circles) and the 50% effective concentration (EC₅₀) at which the drug inhibits SARS-CoV-2-induced cytotoxicity (black squares) as determined by non-linear regression for each drug is shown in (FIGS. 2E-2H). The calculated CC₅₀ and EC₅₀ are shown when appropriate. Data points were obtained from triplicate experiments. Similar results were obtained from an infection with MOI of 0.3; however the dynamic range was not as satisfactory. *, p≤0.05: **, p≤0.01; ***, p≤0.001; ****, p≤0.0001.

FIGS. 3A-3C show that sigma ligands AZ66, CM398, and SA4503 significantly reduce viral genome replication and AZ66 potently reduces SARS-CoV-2 plaque formation. (FIG. 3A), SARS-CoV-2 was used to infect Vero E6 monolayers at an MOI of 0.01 in the presence of 50 μg/ml AZ66, 100 μg/ml for each of CM304, CM398 or SA4503, or 1% DMSO in triplicate. After 48 h, cell monolayers were harvested into AVL buffer and RNA was isolated with the QIAamp viral RNA Kit and qPCR was used to enumerate N copies per ml and are presented as genomic equivalents (GE) per ml. The TO DMSO treatment represents the input GE/ml harvested immediately after virus addition to the monolayers. The data is the mean and standard deviation of three experiments. (FIG. 3B). Phospholipidosis in H23 cells was measured after 48 h of treatment with the indicated sigma ligand concentration. One-way ANOVA indicates significant difference compared to the sertraline positive control. (FIG. 3C) Plaque reduction assay showed the sigma ligand AZ66 was highly effective at inhibiting plaque formation by the SARS-CoV-2 virus. The EC₅₀ of AZ66 in this assay was 6.46 μg/ml (15.93 μM). The calculated EC₅₀ of AZ66 by both cytotoxicity assay and plaque reduction assay is well below the published area under the curve (mean 158.22 μg h/ml) in rats following oral dosing of 20 mg/kg and also below the AUC following an intravenous 5 mg/kg dose (mean 63.2 μg h/ml). *, p≤0.05: ****, p≤0.0001.

FIGS. 4A-4E show that sigma ligands reduce SARS-CoV-2 induced cytopathic effects in cell monolayers. Monolayers of Vero E6 cells in 96-well plates were imaged after 72 h in the absence of treatment (FIG. 4A) or in the presence of AZ66 (FIG. 4B). CM304 (FIG. 4C), CM398 (FIG. 4D), or SA4503 (FIG. 4E) alone. In the bottom row are images of monolayers infected with SARS-CoV-2 at an MOI of 0.2 in the presence of the same drug treatments as in the top panel. Cytopathic effects (CPE) in the monolayers caused by SARS-CoV-2 infection are visible as dark puncta (dead infected cells) against the light-colored intact monolayer. These puncta are absent from the uninfected monolayer images (top row) and are greatly reduced in number in the AZ66 and CM398-treated infected monolayers (FIGS. 4B and 4D: bottom row) compared to untreated infected monolayers (FIG. 4A, bottom row). The scale bar at lower right is equal to 500 μm.

FIGS. 5A-5B show a comparison of agonist versus antagonist interactions with the sigma-1 receptor by molecular docking. The crystal structure of the human sigma-1 receptor (PDB 5HK1) was used as the basis for molecular docking with a selective agonist SA4503 (cutamesine, active against SARS-CoV-2), (FIG. 5A), and antagonist CM304 (inactive against SARS-CoV-2) (FIG. 5B). SA4503 and CM304 are shown as sticks.

FIGS. 6A-6B, show homology modeling of the human sigma-2 receptor and definition of a putative ligand binding site. (FIG. 6A), the crystal structure of Emopamil-Binding Protein (EBP), PDB 60HT, shown in gray, was solved complexed to an inhibitor, UI8666A, shown as spheres. Ligand binding residues are shown as sticks. (FIG. 6B), homology model of the human sigma-2 receptor based on EBP, shown in grey. AZ66, a dual sigma-1 and sigma-2 receptor ligand, is shown as posed by molecular docking using AutoDock Vina as spheres. The putative contact residues on the sigma-2 receptor are shown as sticks.

FIGS. 7A-7B shows molecular docking of sigma-2 receptor ligands that exhibit antiviral activity against SARS-CoV-2. (FIG. 7A), highly selective sigma-2 receptor agonist CM398 is shown as posed by Auto-Dock Vina to a model of the human sigma-2 receptor. (FIG. 7B), dual sigma-1 and sigma-2 receptor ligand AZ66 is shown as posed by molecular docking. Putative interacting residues are shown. CM398 and AZ66 are shown as sticks.

FIGS. 8A-8F show that combinations of diphenhydramine and lactoferrin exhibit synergy against SARS-CoV-2. (FIG. 8A), Vero E6 cells were treated with diphenhydramine (DPH) at various concentrations without (black bars) or with SARS-CoV-2 at MOI 0.2 (gray bars) and cytotoxicity was measured by LDH release. (FIG. 8B). The EC₅₀ (empty circles) and CC₅₀ (black circles) curves were determined by non-linear regression. The EC₅₀ of diphenhydramine alone was 122 μg/ml. (FIG. 8C), Vero E6 cells were treated with diphenhydramine at various concentrations and lactoferrin (LFN) at 400 μg/ml without (black bars) or with SARS-CoV-2 at MOI 0.2 (gray bars) and cytotoxicity was measured by LDH release. (FIG. 8D), The EC₅₀ (empty circles) and CC₅₀ (black circles) curves were determined by non-linear regression. The EC₅₀ of diphenhydramine with 400 μg/ml of lactoferrin was 54.25 μg/ml. (FIG. 8E), The EC₅₀ curves of DPH (empty circles), LFN (black diamonds), and DPH+LFN (black squares) are shown on the same graph to compare effect of LFN on DPH EC₅₀. (FIG. 8F), Measurement of viral genome equivalents by RT-qPCR of the SARS-CoV-2 N-protein gene demonstrate the ability of DPH+LFN to inhibit replication by almost 3-logs. *, p≤0.05; **, p≤0.01: ***, p≤0.001; ****, p≤0.0001; ns, not significant.

FIGS. 9A-9D show inhibition of SARS-CoV-2 infection in human lung epithelial cells. (FIG. 9A) CPE in infected NCI-H23 and NCI-H23ACE2 cells. NCI-H23 (parental untransduced cells), NCI-H23ACE2 pool (lentivirus transformed cells uncloned), and NCI-H23ACE2 (clone A2) were infected with SARS-CoV-2 and CPE was observed 3 dpi. CPE is defined by cell rounding and detachment from the monolayer. The scale bar is equivalent to 100 μm. (FIG. 9B), TCID₅₀ experiments were performed in biological duplicate three times after infecting cells at an MOI of 0.01 for 72 hours. SARS-CoV-2 infection of the human lung epithelial cell line H23 is dependent on heterologous expression of the human ACE2 receptor. (FIG. 9C), The mixed affinity sigma-1/sigma-2 receptor ligand AZ66 and the sigma-2 receptor specific ligand CM398, significantly reduce the amount of infectious SARS-CoV-2 particles released from H23-hACE2 cells by ˜3-log and 1-log, respectively. Data are from TCID₅₀s carried out in technical triplicate. (FIG. 9D), Diphenhydramine and diphenhydramine with lactoferrin significantly reduce infectious SARS-CoV-2 particle release from H23-hACE2 cells by ˜2-log compared to untreated H23-hACE2 cells. Data are from TCID₅₀s carried out in technical triplicate. *, p≤0.05; ****, p≤0.0001; ns, not significant.

FIGS. 10A-10C compare the effects of either saline, 45 mg/kg of AZ66, or 45 mg/kg of CM398 on 6-8 week old K18-hACE2 mice infected with 2.66×104 PFU of SARS-CoV-2 by the intranasal route. (FIG. 10A), % starting mass over time for mice administered saline, AZ66, or CM398. (FIG. 10B), Probability of survival over time for mice administered saline or AZ66. (FIG. 10C), Probability of survival over time for mice administered saline or CM398.

FIGS. 11A-11J show sigma ligands reduce SARS-CoV-2 induced cytopathic effects in cell monolayers. Monolayers of Vero E6 cells in 96-well plates were imaged after 72 hours in the absence of treatment (FIG. 11A) or in the presence of AZ66 (FIG. 11B), CM304 (FIG. 11C), CM398 (FIG. 11D), or SA4503 (FIG. 11E) alone (top row). In the bottom row are images of monolayers infected with SARS-CoV-2 at an MOI of 0.2:1 in the presence of the same drug treatments as in the top panel. Cytopathic effects (CPE) in the monolayers caused by SARS-CoV-2 infection are visible as dark puncta against the light colored intact monolayer: Black dots indicate areas of cytoplasmic effects (CPE) caused by the virus, contrast and brightness increased evenly over all images to aid visualization. These puncta are absent from the uninfected monolayer images (top panel) and are greatly reduced in number in the AZ66 and CM398-treated infected monolayers (FIGS. 11B and 11D; bottom row) compared to untreated infected monolayers (FIG. 11A, bottom row) FIGS. 11F-11J depict raw images of the effect of test compounds (e.g., sigma receptor ligands) on cytopathic effects caused by SARS-CoV-2 described in FIGS. 11A-11E.

### DEFINITIONS

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Pharmaceutically acceptable salts are well known in the art. For example, Berge et al. describe pharmaceutically acceptable salts in detail in J. Pharmaceutical Sciences, 1977, 66, 1-19, incorporated herein by reference.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As defined herein, “SARS-CoV-2” refers to Severe Acute Respiratory Syndrome Coronavirus 2. SARS-CoV-2 has also been known as 2019-nCoV and Wuhan coronavirus. The SARS-CoV-2 genome contains nine genes. Four genes encode for four structural proteins. S (spike), E (envelope), M (matrix) and N (nucleocapsid). The five other genes are orfla, orflab, 3a, 8b, and 7a. SARS-CoV-2 is associated with the nucleotide sequence with a GenBank Accession No.: MN996527.1. SARS-CoV-2 causes the infectious respiratory illness Coronavirus disease 2019 (COVID-19), which is characterized by symptoms including, but not limited to, fever, chills, cough, shortness of breath, difficulty breathing, fatigue, muscle aches, body aches, headache, loss of smell, loss of taste, sore throat, congestion, rhinorrhea, nausea, vomiting, diarrhea, chest pain or pressure, confusion, inability to wake, inability to stay awake, or discolored skin, lips, or nail beds, or any combination thereof.

The term “lung disease” or “pulmonary disorder” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

The term “cardiovascular disorder” refers to diseases and disorders of the heart and circulatory system. Exemplary cardiovascular diseases, including cholesterol- or lipid-related disorders, include, but are not limited to acute coronary syndrome, angina, arrhythmia, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's diseases and inflammatory diseases.

The term “viral infection” generally encompasses infection of an animal host, particularly a human host, by one or more viruses. In some embodiments, the viral infection is caused by a coronavirus, poliovirus, influenza virus, human papillomavirus, human immunodeficiency virus, hepatitis virus, enterovirus, coxsackievirus, bovine ephemeral fever virus, chlorovirus, avian reovirus, or polyomavirus virus. In some embodiments, the viral infection is hepatitis C virus, HIV-1, human papillomavirus 16, influenza A virus, influenza B virus, influenza C virus, poliovirus, respiratory syncytial virus, or SARS-CoV (e.g., SARS-CoV-2) infection. In some embodiments, there is clinical evidence of the infection based on symptoms or based on the demonstration of the presence of the viral pathogen in a biological sample from the host or subject.

The term “respiratory disorder” refers to any condition and/or disorder relating to respiration and/or the respiratory system, or a portion thereof. In some embodiments, the respiratory tract can refer to the respiratory tract of a human or a non-human mammal. In some embodiments, the term “respiratory disorder” refers to a disease or disorder characterized by excessive mucus production in the pulmonary airways (e.g., cystic fibrosis), or defective ciliary function (e.g., primary ciliary dyskinesia syndrome), respiratory dysfunction, obstructive lung disease (e.g., chronic bronchitis, chronic obstructive pulmonary disease (COPD), emphysema) or reduced pulmonary function (e.g., atelectasis, pneumothorax, bronchial asthma).

### DETAILED DESCRIPTION OF CERTAIN EMBODIMENTS

Provided herein are compounds, and salts thereof, and pharmaceutical compositions and kits thereof. The compounds provided herein have sigma receptor binding affinity (e.g., sigma-1 receptor binding affinity or sigma-2 receptor binding affinity). Also provided herein are methods of treating and/or preventing SARS-CoV-2 infection, symptoms of SARS-CoV-2 infection, or dysfunction associated with SARS-CoV-2 infection in a subject comprising administration to the subject of a compound, or salt thereof, having sigma receptor binding affinity. Other uses of the compounds provided herein include methods for reducing replication of SARS-CoV-2 virus.

### Pharmaceutical Compositions, Kits, and Administration

The present disclosure provides compositions comprising a compound that is AZ66, CM304, CM398, or SA4503, or a salt thereof, and a pharmaceutically acceptable excipient. In certain embodiments, the composition described herein comprises a compound that is AZ66, CM304, CM398, or SA4503, or a salt thereof, and a pharmaceutically acceptable excipient. In some embodiments, the composition described herein comprises a compound that is AZ66, or a salt thereof, and a pharmaceutically acceptable excipient. In certain embodiments, the composition described herein comprises a compound that is CM304, or a salt thereof, and a pharmaceutically acceptable excipient. In some embodiments, the composition described herein comprises a compound that is CM398, or a salt thereof, and a pharmaceutically acceptable excipient. In certain embodiments, the composition described herein comprises a compound that is SA4503, or a salt thereof, and a pharmaceutically acceptable excipient.

In certain embodiments, the composition is for oral, nasal, or injectable administration. In some embodiments, the composition is for oral or nasal administration. In certain embodiments, the composition is for oral or injectable administration. In some embodiments, the composition is for nasal or injectable administration. In certain embodiments, the composition is for oral administration. In some embodiments, the composition is for nasal administration. In certain embodiments, the composition is for injectable administration.

In some embodiments, a composition provided herein further comprises an additional therapeutic agent. In some embodiments, the additional therapeutic agent is an antiviral agent, an immunomodulatory agent, an immunosuppressant, an anti-inflammatory agent, an antibody. In some embodiments, the additional therapeutic agent is an antiviral agent. In some embodiments, the additional therapeutic agent is an immunomodulatory agent. In some embodiments, the additional therapeutic agent is an immunosuppressant. In some embodiments, the additional therapeutic agent is an anti-inflammatory agent. In some embodiments, the additional therapeutic agent is an antibody.

In certain embodiments, the composition is a pharmaceutical composition.

In certain embodiments, the compound described herein is provided in an effective amount in the pharmaceutical composition. In certain embodiments, the effective amount is a therapeutically effective amount. In certain embodiments, a therapeutically effective amount is an amount sufficient for reducing replication of SARS-CoV-2 virus. In certain embodiments, a therapeutically effective amount is an amount sufficient for treating SARS-Cov-2. In certain embodiments, a therapeutically effective amount is an amount sufficient for reducing replication of SARS-CoV-2 virus and treating SARS-Cov-2. In certain embodiments, a therapeutically effective amount is an amount sufficient for treating symptoms of SARS-Cov-2. In certain embodiments, a therapeutically effective amount is an amount sufficient for reducing replication of SARS-CoV-2 virus and treating symptoms of SARS-Cov-2. In certain embodiments, a therapeutically effective amount is an amount sufficient for treating respiratory symptoms of SARS-Cov-2. In certain embodiments, a therapeutically effective amount is an amount sufficient for reducing replication of SARS-CoV-2 virus and treating respiratory symptoms of SARS-Cov-2. In certain embodiments, a therapeutically effective amount is an amount sufficient for treating pulmonary dysfunction associated with SARS-Cov-2. In certain embodiments, a therapeutically effective amount is an amount sufficient for reducing replication of SARS-CoV-2 virus and treating pulmonary dysfunction associated with SARS-Cov-2. In certain embodiments, a therapeutically effective amount is an amount sufficient for treating cardiovascular dysfunction associated with SARS-Cov-2. In certain embodiments, a therapeutically effective amount is an amount sufficient for reducing replication of SARS-CoV-2 virus and treating cardiovascular dysfunction associated with SARS-Cov-2. In certain embodiments, a therapeutically effective amount is an amount sufficient for treating metabolic dysfunction associated with SARS-Cov-2. In certain embodiments, a therapeutically effective amount is an amount sufficient for reducing replication of SARS-CoV-2 virus and treating metabolic dysfunction associated with SARS-CoV-2.

In certain embodiments, the effective amount is a prophylactically effective amount. In certain embodiments, a prophylactically effective amount is an amount sufficient for reducing replication of SARS-CoV-2 virus and preventing SARS-Cov-2. In certain embodiments, a prophylactically effective amount is an amount sufficient for preventing symptoms of SARS-Cov-2. In certain embodiments, a prophylactically effective amount is an amount sufficient for reducing replication of SARS-CoV-2 virus and preventing symptoms of SARS-Cov-2. In certain embodiments, a prophylactically effective amount is an amount sufficient for preventing respiratory symptoms of SARS-Cov-2. In certain embodiments, a prophylactically effective amount is an amount sufficient for reducing replication of SARS-CoV-2 virus and preventing respiratory symptoms of SARS-Cov-2. In certain embodiments, a prophylactically effective amount is an amount sufficient for preventing pulmonary dysfunction associated with SARS-Cov-2. In certain embodiments, a prophylactically effective amount is an amount sufficient for reducing replication of SARS-CoV-2 virus and preventing pulmonary dysfunction associated with SARS-Cov-2. In certain embodiments, a prophylactically effective amount is an amount sufficient for preventing cardiovascular dysfunction associated with SARS-Cov-2. In certain embodiments, a prophylactically effective amount is an amount sufficient for reducing replication of SARS-CoV-2 virus and preventing cardiovascular dysfunction associated with SARS-Cov-2. In certain embodiments, a prophylactically effective amount is an amount sufficient for preventing metabolic dysfunction associated with SARS-Cov-2. In certain embodiments, a prophylactically effective amount is an amount sufficient for reducing replication of SARS-CoV-2 virus and preventing metabolic dysfunction associated with SARS-Cov-2.

In some embodiments, the effective amount of the compound is about between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive. In certain embodiments, the effective amount includes independently between 1 mg and 3 mg, inclusive, of a compound described herein. In certain embodiments, the effective amount described herein includes independently between 3 mg and 10 mg, inclusive, of a compound described herein. In certain embodiments, the effective amount described herein includes independently between 10 mg and 100 mg, inclusive, of a compound described herein. In certain embodiments, the effective amount described herein includes independently between 10 mg and 30 mg, inclusive, of a compound described herein. In certain embodiments, the effective amount includes independently between 30 mg and 100 mg, inclusive, of a compound described herein.

In certain embodiments, the effective amount is about 0.1 mg/kg to about 200 mg/kg of the compound. In some embodiments, the effective amount is about 0.1 mg/kg to about 150 mg/kg of the compound. In certain embodiments, the effective amount is about 0.1 mg/kg to about 100 mg/kg of the compound. In some embodiments, the effective amount is about 0.1 mg/kg to about 75 mg/kg of the compound. In certain embodiments, the effective amount is about 0.1 mg/kg to about 60 mg/kg of the compound. In some embodiments, the effective amount is about 1 mg/kg to about 200 mg/kg of the compound. In certain embodiments, the effective amount is about 1 mg/kg to about 150 mg/kg of the compound. In some embodiments, the effective amount is about 1 mg/kg to about 100 mg/kg of the compound. In certain embodiments, the effective amount is about 1 mg/kg to about 75 mg/kg of the compound. In some embodiments, the effective amount is about 1 mg/kg to about 60 mg/kg of the compound. In certain embodiments, the effective amount is about 0.1 mg/kg, about 0.5 mg/kg, about 1 mg/kg, about 5 mg/kg, about 10 mg/kg, about 20 mg/kg, about 30 mg/kg of the compound, about 35 mg/kg, about 40 mg/kg, about 45 mg/kg, about 50 mg/kg, about 60 mg/kg, about 65 mg/kg, about 70 mg/kg, about 75 mg/kg, about 80 mg/kg, about 90 mg/kg, about 100 mg/kg, about 110 mg/kg, about 120 mg/kg, about 130 mg/kg, about 140 mg/kg, about 150 mg/kg, or about 200 mg/kg.

In some embodiments, the effective amount has a concentration of the compound of about between 0.001 μM and 0.01 μM, 0.01 μM and 0.1 μM, between 0.1 μM and 1 μM, between 1 μM and 10 μM, between 10 μM and 50 μM, between 50 μM and 100 μM, or between 100 and 500 μM, inclusive. In some embodiments, the effective amount has a concentration of the compound of about between 0.001 μM and 0.01 μM, inclusive, of a compound described herein. In certain embodiments, the effective amount includes independently between 0.01 μM and 0.1 μM, inclusive, of a compound described herein. In certain embodiments, the effective amount includes independently between 0.1 μM and 1 μM, inclusive, of a compound described herein. In certain embodiments, the effective amount includes independently between 1 μM and 10 μM, inclusive, of a compound described herein. In certain embodiments, the effective amount includes independently between 10 μM and 50 μM, inclusive, of a compound described herein. In certain embodiments, the effective amount includes independently between 50 μM and 100 μM, inclusive, of a compound described herein. In certain embodiments, the effective amount includes independently between 100 μM and 500 μM, inclusive, of a compound described herein.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer) carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® 11, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate. D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia. (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65 CF at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed: the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual: by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., viral infection, respiratory disorder, pulmonary disorder, cardiovascular disorder, inflammatory disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs. RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, provided herein are kits including a first container comprising a compound or pharmaceutical composition described herein. In certain embodiments, the kits are useful for treating SARS-CoV-2 or symptoms thereof in a subject in need thereof. In certain embodiments, the kits are useful for preventing SARS-CoV-2 or symptoms thereof in a subject in need thereof. In certain embodiments, the kits are useful for reducing the risk of developing SARS-CoV-2 or symptoms thereof in a subject in need thereof. In certain embodiments, the kits are useful for reducing replication of SARS-CoV-2 virus in a subject.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating SARS-CoV-2 or symptoms thereof in a subject in need thereof. In certain embodiments, the kits and instructions provide for preventing SARS-CoV-2 or symptoms thereof in a subject in need thereof. In certain embodiments, the kits and instructions provide for reducing the risk of developing SARS-CoV-2 or symptoms thereof in a subject in need thereof. In certain embodiments, the kits and instructions provide for reducing replication of SARS-CoV-2 virus in a subject. A kit described herein may include one or more additional pharmaceutical agents described herein as a separate composition.

### Methods of Treatment and Uses

Provided herein are methods for treating and/or preventing SARS-CoV-2 infection in a subject, the methods comprising administering a compound having sigma receptor binding affinity (e.g., compounds herein). In some embodiments, the methods are for treating or preventing SARS-CoV-2 infection in a subject. In some embodiments, the methods are for treating or preventing symptoms of SARS-CoV-2 infection in a subject. In some embodiments, the methods are for treating or preventing respiratory symptoms of SARS-CoV-2 infection in a subject. In some embodiments, the methods are for treating or preventing pulmonary dysfunction associated with SARS-CoV-2 infection in a subject. In some embodiments, the methods are for treating or preventing cardiovascular dysfunction associated with SARS-CoV-2 infection in a subject. In some embodiments, the methods are for treating or preventing metabolic dysfunction associated with SARS-CoV-2 infection in a subject. In some embodiments, the methods comprise administering a therapeutically effective amount of the compound to a subject. In some embodiments, the methods comprise administering a therapeutically effective amount of the compound to a subject identified as in need of administration of the compound. In some embodiments, the methods comprise administering a prophylactically effective amount of the compound to a subject. In some embodiments, the methods comprise administering a prophylactically effective amount of the compound to a subject identified as in need of administration of the compound.

Also provided herein are uses of compounds, and salts thereof, having sigma receptor binding affinity and pharmaceutical compositions thereof, for the preparation of a medicament. In some embodiments, the medicament is for treating or preventing SARS-CoV-2 infection in a subject. In some embodiments, the medicament is for treating or preventing symptoms of SARS-CoV-2 infection in a subject. In some embodiments, the medicament is for treating or preventing respiratory symptoms of SARS-CoV-2 infection in a subject. In some embodiments, the medicament is for treating or preventing pulmonary dysfunction associated with SARS-CoV-2 infection in a subject. In some embodiments, the medicament is for treating or preventing cardiovascular dysfunction associated with SARS-CoV-2 infection in a subject. In some embodiments, the medicament is for treating or preventing metabolic dysfunction associated with SARS-CoV-2 infection in a subject.

Also provided herein are compounds, and salts thereof, having sigma receptor binding affinity, and pharmaceutical composition thereof, for use in treating and/or preventing SARS-CoV-2 in a subject. In some embodiments, the compound, or a salt thereof, having sigma receptor binding affinity, and pharmaceutical composition thereof, is for use in treating or preventing symptoms of SARS-CoV-2 infection in a subject. In some embodiments, the compound, or a salt thereof, having sigma receptor binding affinity, and pharmaceutical composition thereof, is for use in treating or preventing respiratory symptoms of SARS-CoV-2 infection in a subject. In some embodiments, the compound, or a salt thereof, having sigma receptor binding affinity, and pharmaceutical composition thereof, is for use in treating or preventing pulmonary dysfunction associated with SARS-CoV-2 infection in a subject. In some embodiments, the compound, or a salt thereof, having sigma receptor binding affinity, and pharmaceutical composition thereof, is for use in treating or preventing cardiovascular dysfunction associated with SARS-CoV-2 infection in a subject. In some embodiments, the compound, or a salt thereof, having sigma receptor binding affinity, and pharmaceutical composition thereof, is for use in treating or preventing metabolic dysfunction associated with SARS-CoV-2 infection in a subject.

Also provided herein are methods for reducing replication of SARS-CoV-2 virus in a subject, comprising administration to the subject a compound, or salt thereof, having sigma receptor binding affinity.

In some embodiments, replication of SARS-CoV-2 virus is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97.5%, at least 99%, or at least 99.9%. In some embodiments, replication of SARS-CoV-2 virus is reduced by at least 50%. In some embodiments, replication of SARS-CoV-2 virus is reduced by at least 55%. In some embodiments, replication of SARS-CoV-2 virus is reduced by at least 60%. In some embodiments, replication of SARS-CoV-2 virus is reduced by at least 65%. In some embodiments, replication of SARS-CoV-2 virus is reduced by at least 70%. In some embodiments, replication of SARS-CoV-2 virus is reduced by at least 75%. In some embodiments, replication of SARS-CoV-2 virus is reduced by at least 80%, In some embodiments, replication of SARS-CoV-2 virus is reduced by at least 85%. In some embodiments, replication of SARS-CoV-2 virus is reduced by at least 90%. In some embodiments, replication of SARS-CoV-2 virus is reduced by at least 95%. In some embodiments, replication of SARS-CoV-2 virus is reduced by at least 97.5%. In some embodiments, replication of SARS-CoV-2 virus is reduced by at least 99%. In some embodiments, replication of SARS-CoV-2 virus is reduced by at least 99.9%.

In some embodiments, replication of SARS-CoV-2 virus is reduced by 0-10%, 5-15%, 10-20%, 15-25%, 20-30%, 25-35%, 30-40%, 35-35%, 40-50%, 45-55%, 50-60%, 55-65%, 60-70%, 65-75%, 70-80%, 75-85%, 80-90%, 85-95%, or 90-100%. In some embodiments, replication of SARS-CoV-2 virus is reduced by 44-55%, 50-60%, 55-65%, 60-70%, 65-75%, 70-80%, 75-85%, 80-90%, 85-95%, or 90-100%. In some embodiments, replication of SARS-CoV-2 virus is reduced by 45-55%. In some embodiments, replication of SARS-CoV-2 virus is reduced by 50-60%. In some embodiments, replication of SARS-CoV-2 virus is reduced by 55-65%. In some embodiments, replication of SARS-CoV-2 virus is reduced by 60-70%. In some embodiments, replication of SARS-CoV-2 virus is reduced by 65-75%. In some embodiments, replication of SARS-CoV-2 virus is reduced by 70-80%. In some embodiments, replication of SARS-CoV-2 virus is reduced by 75-85%. In some embodiments, replication of SARS-CoV-2 virus is reduced by 80-90%. In some embodiments, replication of SARS-CoV-2 virus is reduced by 85-95%. In some embodiments, replication of SARS-CoV-2 virus is reduced by 90-100%.

In some embodiments, replication of SARS-CoV-2 virus is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97.5%, about 99%, or about 99.9%. In some embodiments, replication of SARS-CoV-2 virus is reduced by about 50%. In some embodiments, replication of SARS-CoV-2 virus is reduced by about 55%. In some embodiments, replication of SARS-CoV-2 virus is reduced by about 60%. In some embodiments, replication of SARS-CoV-2 virus is reduced by about 65%. In some embodiments, replication of SARS-CoV-2 virus is reduced by about 70%. In some embodiments, replication of SARS-CoV-2 virus is reduced by about 75%.

In some embodiments, replication of SARS-CoV-2 virus is reduced by about 80%, In some embodiments, replication of SARS-CoV-2 virus is reduced by about 85%. In some embodiments, replication of SARS-CoV-2 virus is reduced by about 90%. In some embodiments, replication of SARS-CoV-2 virus is reduced by about 95%. In some embodiments, replication of SARS-CoV-2 virus is reduced by about 97.5%. In some embodiments, replication of SARS-CoV-2 virus is reduced by about 99%. In some embodiments, replication of SARS-CoV-2 virus is reduced by about 99.9%.

Also provided herein are methods for reducing SARS-CoV-2 virus-induced cellular toxicity in a subject, comprising administration to the subject of a compound, or salt thereof, having sigma receptor binding affinity.

In some embodiments, the compound inhibits cytopathic effects. In some embodiments, the compound reduces cytopathic effects. In some embodiments, the compound has selective antiviral activity.

In some embodiments, the cytotoxicity of ≤50 μg/mL of the compound in SARS-CoV-2 infected cells is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97.5%, at least 99%, or at least 99.9%. In some embodiments, the cytotoxicity of ≤50 μg/mL of the compound in SARS-CoV-2 infected cells is at least 40%. In some embodiments, the cytotoxicity of ≤50 μg/mL of the compound in SARS-CoV-2 infected cells is at least 45%. In some embodiments, the cytotoxicity of ≤50 μg/mL of the compound in SARS-CoV-2 infected cells is at least 50%. In some embodiments, the cytotoxicity of ≤50 μg/mL of the compound in SARS-CoV-2 infected cells is at least 55%. In some embodiments, the cytotoxicity of ≤50 μg/mL of the compound in SARS-CoV-2 infected cells is at least 60%. In some embodiments, the cytotoxicity of ≤50 μg/mL of the compound in SARS-CoV-2 infected cells is at least 65%. In some embodiments, the cytotoxicity of ≤50 μg/mL of the compound in SARS-CoV-2 infected cells is at least 70%. In some embodiments, the cytotoxicity of ≤50 μg/mL of the compound in SARS-CoV-2 infected cells is at least 75%. In some embodiments, the cytotoxicity of ≤50 μg/mL of the compound in SARS-CoV-2 infected cells is at least 80%, In some embodiments, the cytotoxicity of ≤50 μg/mL of the compound in SARS-CoV-2 infected cells is at least 85%. In some embodiments, the cytotoxicity of ≤50 μg/mL of the compound in SARS-CoV-2 infected cells is at least 90%. In some embodiments, the cytotoxicity of ≤50 μg/mL of the compound in SARS-CoV-2 infected cells is at least 95%. In some embodiments, the cytotoxicity of ≤50 μg/mL of the compound in SARS-CoV-2 infected cells is at least 97.5%. In some embodiments, the cytotoxicity of ≤50 μg/mL of the compound in SARS-CoV-2 infected cells is at least 99%. In some embodiments, the cytotoxicity of ≤50 μg/mL of the compound in SARS-CoV-2 infected cells is at least 99.9%.

In some embodiments, the compound has a selectivity index (CC₅₀/EC₅₀) of at least 0.05, at least 0.1, at least 0.25, at least 0.5, at least 0.75, at least 1.0, at least 1.25, at least 1.5, at least 1.75, at least 2.0, at least 2.25, at least 2.5, or at least 2.75 as measured by cytotoxicity. In some embodiments, the compound has a selectivity index (CC₅₀/EC₅₀) of at least 0.05 as measured by cytotoxicity. In some embodiments, the compound has a selectivity index (CC₅₀/EC₅₀) of at least 0.1 as measured by cytotoxicity. In some embodiments, the compound has a selectivity index (CC₅₀/EC₅₀) of at least 0.25 as measured by cytotoxicity. In some embodiments, the compound has a selectivity index (CC₅₀/EC₅₀) of at least 0.5 as measured by cytotoxicity. In some embodiments, the compound has a selectivity index (CC₅₀/EC₅₀) of at least 0.75 as measured by cytotoxicity. In some embodiments, the compound has a selectivity index (CC₅₀/EC₅₀) of at least 1.0 as measured by cytotoxicity. In some embodiments, the compound has a selectivity index (CC₅₀/EC₅₀) of at least 1.25 as measured by cytotoxicity. In some embodiments, the compound has a selectivity index (CC₅₀/EC₅₀) of at least 1.5 as measured by cytotoxicity. In some embodiments, the compound has a selectivity index (CC₅₀/EC₅₀) of at least 1.75 as measured by cytotoxicity. In some embodiments, the compound has a selectivity index (CC₅₀/EC₅₀) of at least 2.0 as measured by cytotoxicity. In some embodiments, the compound has a selectivity index (CC₅₀/EC₅₀) of at least 2.25 as measured by cytotoxicity. In some embodiments, the compound has a selectivity index (CC₅₀/EC₅₀) of at least 2.5 as measured by cytotoxicity. In some embodiments, the compound has a selectivity index (CC₅₀/EC₅₀) of at least 2.75 as measured by cytotoxicity.

In some embodiments, the compound has a selectivity index (CC₅₀/EC₅₀) of at least 2.5, at least 5.0, at least 7.5, at least 10.0, at least 12.5, at least 15.0, at least 17.5, at least 18.0, or at least 19.0 as measured by plaque assay. In some embodiments, the compound has a selectivity index (CC₅₀/EC₅₀) of at least 2.5 as measured by plaque assay. In some embodiments, the compound has a selectivity index (CC₅₀/EC₅₀) of at least 5.0 as measured by plaque assay. In some embodiments, the compound has a selectivity index (CC₅₀/EC₅₀) of at least 7.5 as measured by plaque assay. In some embodiments, the compound has a selectivity index (CC₅₀/EC₅₀) of at least 10.0 as measured by plaque assay. In some embodiments, the compound has a selectivity index (CC₅₀/EC₅₀) of at least 12.5 as measured by plaque assay. In some embodiments, the compound has a selectivity index (CC₅₀/EC₅₀) of at least 15.0 as measured by plaque assay. In some embodiments, the compound has a selectivity index (CC₅₀/EC₅₀) of at least 17.5 as measured by plaque assay. In some embodiments, the compound has a selectivity index (CC₅₀/EC₅₀) of at least 18.0 as measured by plaque assay. In some embodiments, the compound has a selectivity index (CC₅₀/EC₅₀) of at least 19.0 as measured by plaque assay.

In some embodiments, the compound inhibits plaque formation caused by SARS-CoV-2.

In some embodiments, the compound delays weight loss associated with SARS-CoV-2 infection. In some embodiments, the weight loss is reduced over a period of 1 day, 2 days, 3 days, 4 days, 5 days, 6 days, 7 days, or two weeks. In some embodiments, the weight loss is reduced over a period of about 1 day. In some embodiments, the weight loss is reduced over a period of about 2 days. In some embodiments, the weight loss is reduced over a period of about 3 days. In some embodiments, the weight loss is reduced over a period of about 4 days. In some embodiments, the weight loss is reduced over a period of about 5 days. In some embodiments, the weight loss is reduced over a period of about 6 days. In some embodiments, the weight loss is reduced over a period of about 7 days. In some embodiments, the weight loss is reduced over a period of about two weeks.

In some embodiments, the compound reduces weight loss associated with SARS-CoV-2 infection. In some embodiments, the compound reduces weight loss by at least 1%, at least 2.5%, at least 5%, at least 10%, or at least 15% of starting mass. In some embodiments, the compound reduces weight loss by at least 1% of starting mass. In some embodiments, the compound reduces weight loss by at least 2.5% of starting mass. In some embodiments, the compound reduces weight loss by at least 5% of starting mass. In some embodiments, the compound reduces weight loss by at least 10% of starting mass.

In some embodiments, the compound reduces weight loss by about 1%, about 2.5%, about 5%, about 10%, about 15%, or about 20% of starting mass. In some embodiments, the compound reduces weight loss by about 1% of starting mass. In some embodiments, the compound reduces weight loss by about 2.5% of starting mass. In some embodiments, the compound reduces weight loss by about 5% of starting mass. In some embodiments, the compound reduces weight loss by about 10% of starting mass. In some embodiments, the compound reduces weight loss by about 15% of starting mass.

In some embodiments, the compound promotes recovery of weight lost due to SARS-CoV-2 infection. In some embodiments, the compound promotes return to about 85%, about 90%, about 95%, about 97.5%, about 99%, about 99.5%, about 99.9%, or about 100% of starting mass prior to weight loss associated with to SARS-CoV-2 infection. In some embodiments, the compound promotes return to about 85% of starting mass prior to weight loss associated with to SARS-CoV-2 infection. In some embodiments, the compound promotes return to about 90% of starting mass prior to weight loss associated with to SARS-CoV-2 infection. In some embodiments, the compound promotes return to about 95% of starting mass prior to weight loss associated with to SARS-CoV-2 infection. In some embodiments, the compound promotes return to about 97.5% of starting mass prior to weight loss associated with to SARS-CoV-2 infection. In some embodiments, the compound promotes return to about 99% of starting mass prior to weight loss associated with to SARS-CoV-2 infection. In some embodiments, the compound promotes return to about 99.5% of starting mass prior to weight loss associated with to SARS-CoV-2 infection. In some embodiments, the compound promotes return to about 99.9% of starting mass prior to weight loss associated with to SARS-CoV-2 infection. In some embodiments, the compound promotes return to about 100% of starting mass prior to weight loss associated with to SARS-CoV-2 infection.

In some embodiments, the subject is a mammal. In some embodiments, the subject is a rodent. In some embodiments, the subject is a mouse. In some embodiments, the subject is a human. In some embodiments, the subject is less than 18 years of age. In some embodiments, the subject is a human. In some embodiments, the subject is greater than 18 years of age. In some embodiments, the subject is 18-65 years of age. In some embodiments, the subject is less than 65 years of age. In some embodiments, the subject is greater than 65 years of age.

In some embodiments, the subject has been identified as having SARS-CoV-2 infection. In some embodiments, the subject has SARS-CoV-2 infection. In some embodiments, the subject has been identified as in need of such treatment. In some embodiments, the subject is in need of such treatment. In some embodiments, the subject is administered a therapeutically effective amount of compound, or salt thereof. In some embodiments, the subject has been diagnosed with COVID-19. In some embodiments, the subject has been diagnosed with COVID-19 by a molecular amplification test. In some embodiments, the subject has been diagnosed with COVID-19 by a PCR test. In some embodiments, the subject has been diagnosed with COVID-19 by an antigen test. In some embodiments, the subject has been diagnosed with COVID-19 by an antibody test. In some embodiments, the subject has previously had COVID-19. In some embodiments, the subject has not previously had COVID-19. In some embodiments, the subject exhibits one or more symptoms associated with COVID-19 or SARS-CoV-2 infection. In some embodiments the subject exhibits no symptoms associated with COVID-19 or SARS-CoV-2 infection (asymptomatic).

In some embodiments, the symptoms of SARS-CoV-2 infection are one or more of fever, chills, cough, shortness of breath, difficulty breathing, fatigue, muscle aches, body aches, headache, loss of smell, loss of taste, sore throat, congestion, rhinorrhea, nausea, vomiting, diarrhea, chest pain or pressure, confusion, inability to wake, inability to stay awake, weight loss, or discolored skin, lips, or nail beds. In some embodiments, the symptoms of SARS-CoV-2 infection are one or more of chest pain or pressure, confusion, inability to wake, inability to stay awake, or discolored skin, lips, or nail beds. In some embodiments, the symptom of SARS-CoV-2 infection is chest pain or pressure. In some embodiments, the symptom of SARS-CoV-2 infection is confusion. In some embodiments, the symptom of SARS-CoV-2 infection is inability to wake. In some embodiments, the symptom of SARS-CoV-2 infection is inability to stay awake. In some embodiments, the symptom of SARS-CoV-2 is weight loss. In some embodiments, the symptom of SARS-CoV-2 infection is discolored skin, lips, or nail beds. In some embodiments, the symptoms of SARS-CoV-2 infection are one or more of fever, chills, cough, shortness of breath, difficulty breathing, fatigue, muscle aches, body aches, headache, loss of smell, loss of taste, sore throat, congestion, rhinorrhea, nausea, vomiting, or diarrhea. In some embodiments, the symptoms of SARS-CoV-2 infection are fever or chills. In some embodiments, the symptom of SARS-CoV-2 is fever. In some embodiments, the symptom of SARS-CoV-2 is chills. In some embodiments, the symptom of SARS-CoV-2 is cough. In some embodiments, the symptoms of SARS-CoV-2 infection are shortness of breath or difficulty breathing. In some embodiments, the symptom of SARS-CoV-2 is shortness of breath. In some embodiments, the symptom of SARS-CoV-2 is difficulty breathing. In some embodiments, the symptom of SARS-CoV-2 is fatigue. In some embodiments, the symptoms of SARS-CoV-2 infection are muscle aches or body aches. In some embodiments, the symptom of SARS-CoV-2 is muscle aches. In some embodiments, the symptom of SARS-CoV-2 is body aches. In some embodiments, the symptom of SARS-CoV-2 is headache. In some embodiments, the symptoms of SARS-CoV-2 infection are loss of tase or smell. In some embodiments, the symptom of SARS-CoV-2 is loss of smell. In some embodiments, the symptom of SARS-CoV-2 is loss of taste. In some embodiments, the symptom of SARS-CoV-2 is sore throat. In some embodiments, the symptoms of SARS-CoV-2 infection are congestion or rhinorrhea. In some embodiments, the symptom of SARS-CoV-2 is congestion. In some embodiments, the symptom of SARS-CoV-2 is rhinorrhea. In some embodiments, the symptoms of SARS-CoV-2 infection are nausea or vomiting. In some embodiments, the symptom of SARS-CoV-2 is nausea. In some embodiments, the symptom of SARS-CoV-2 is vomiting. In some embodiments, the symptom of SARS-CoV-2 is diarrhea.

In some embodiments, the respiratory symptoms of SARS-CoV-2 infection are coughing, sputum, shortness of breath, difficulty breathing, or sore throat. In some embodiments, the respiratory symptoms of SARS-CoV-2 infection are coughing, sputum, shortness of breath, or difficulty breathing. In some embodiments, the respiratory symptoms of SARS-CoV-2 infection is coughing. In some embodiments, the respiratory symptoms of SARS-CoV-2 infection is sputum. In some embodiments, the respiratory symptoms of SARS-CoV-2 infection is shortness of breath. In some embodiments, the respiratory symptoms of SARS-CoV-2 infection is difficulty breathing. In some embodiments, the respiratory symptoms of SARS-CoV-2 infection is sore throat.

In some embodiments, the pulmonary dysfunction associated with SARS-CoV-2 infection is interstitial thickening, fibrosis, decreased diffusion capacity for carbon monoxide, or diminished respiratory muscle strength. In some embodiments, the pulmonary dysfunction associated with SARS-CoV-2 infection is interstitial thickening or fibrosis. In some embodiments, the pulmonary dysfunction associated with SARS-CoV-2 infection is decreased diffusion capacity for carbon monoxide or diminished respiratory muscle strength. In some embodiments, the pulmonary dysfunction associated with SARS-CoV-2 infection is interstitial thickening. In some embodiments, the pulmonary dysfunction associated with SARS-CoV-2 infection is fibrosis. In some embodiments, the pulmonary dysfunction associated with SARS-CoV-2 infection is decreased diffusion capacity for carbon monoxide. In some embodiments, the pulmonary dysfunction associated with SARS-CoV-2 infection is diminished respiratory muscle strength.

In some embodiments, the cardiovascular dysfunction associated with SARS-CoV-2 infection is myocardial injury, thromboembolic disease, myocardial inflammation, myocarditis, cardiac arrhythmia, or heart failure. In some embodiments, the cardiovascular dysfunction associated with SARS-CoV-2 infection is myocardial injury. In some embodiments, the cardiovascular dysfunction associated with SARS-CoV-2 infection is thromboembolic disease. In some embodiments, the cardiovascular dysfunction associated with SARS-CoV-2 infection is myocardial inflammation. In some embodiments, the cardiovascular dysfunction associated with SARS-CoV-2 infection is myocarditis. In some embodiments, the cardiovascular dysfunction associated with SARS-CoV-2 infection is cardiac arrhythmia. In some embodiments, the cardiovascular dysfunction associated with SARS-CoV-2 infection is heart failure.

In some embodiments, the metabolic dysfunction associated with SARS-CoV-2 infection is pancreatic function, hyperglycemia, ketoacidosis, diabetes, and metabolic complications of pre-existing diabetes. In some embodiments, the metabolic dysfunction associated with SARS-CoV-2 infection is pancreatic function. In some embodiments, the metabolic dysfunction associated with SARS-CoV-2 infection is hyperglycemia. In some embodiments, the metabolic dysfunction associated with SARS-CoV-2 infection is ketoacidosis. In some embodiments, the metabolic dysfunction associated with SARS-CoV-2 infection is diabetes. In some embodiments, the metabolic dysfunction associated with SARS-CoV-2 infection is metabolic complications of pre-existing diabetes.

In some embodiments, the compound, or salt thereof, exhibits antiviral activity.

In some embodiments, the compound, or salt thereof, has sigma-1 receptor binding affinity. In some embodiments, the compound, or salt thereof, has sigma-1 receptor binding affinity and exhibits antiviral activity. In some embodiments, the compound, or salt thereof, that has sigma-1 receptor binding affinity is SA4503. In some embodiments, the compound, or salt thereof, that has sigma-1 receptor binding affinity is CM304. In some embodiments, the compound, or salt thereof, that has sigma-1 receptor binding affinity is AZ66.

In some embodiments, the compound, or salt thereof, has sigma-2 receptor binding affinity. In some embodiments, the compound, or salt thereof, has sigma-2 receptor binding affinity and exhibits antiviral activity. In some embodiments, the compound, or salt thereof, that has sigma-2 receptor binding affinity is CM398. In some embodiments, the compound, or salt thereof, that has sigma-2 receptor binding affinity is AZ66.

In some embodiments, the compound, or salt thereof, is sigma-1 receptor specific. In some embodiments, the compound, or salt thereof, is sigma-2 receptor specific. In some embodiments, the compound, or salt thereof, has both sigma-1 and sigma-2 receptor binding affinity.

In some embodiments, the compound, or salt thereof, is a sigma receptor agonist. In some embodiments, the compound, or salt thereof, is a sigma receptor agonist and exhibits antiviral activity. In some embodiments, the compound, or salt thereof, is a sigma-1 receptor agonist. In some embodiments, the compound, or salt thereof, is a sigma-1 receptor agonist and exhibits antiviral activity. In some embodiments, the sigma-1 receptor agonist is SA4503. In some embodiments, the compound, or salt thereof, is a sigma-2 receptor agonist. In some embodiments, the compound, or salt thereof, is a sigma-2 receptor agonist and exhibits antiviral activity. In some embodiments, the sigma-2 receptor agonist is CM398.

In some embodiments, the compound, or salt thereof, is a sigma receptor antagonist. In some embodiments, the compound, or salt thereof, is a sigma receptor antagonist and exhibits antiviral activity. In some embodiments, the compound, or salt thereof, is a sigma-1 receptor antagonist. In some embodiments, the compound, or salt thereof, is a sigma-1 receptor antagonist and exhibits antiviral activity. In some embodiments, the sigma-1 receptor antagonist is CM304. In some embodiments, the compound, or salt thereof, is a sigma-2 receptor antagonist. In some embodiments, the compound, or salt thereof, is a sigma-2 receptor antagonist and exhibits antiviral activity.

In some embodiments, the compound has off-target binding sigma receptor binding affinity. In some embodiments, the compound has off-target sigma-1 receptor binding affinity. In some embodiments, the compound that has off-target sigma-1 receptor binding affinity is diphenhydramine.

In some embodiments, the compound, or salt thereof, is AZ66. CM304, CM398, or SA4503, or a salt thereof. In some embodiments, the compound, or salt thereof, is AZ66, or a salt thereof. In certain embodiments, the compound, or salt thereof, is CM304, or a salt thereof. In some embodiments, the compound, or salt thereof, is CM398, or a salt thereof. In certain embodiments, the compound, or salt thereof, is SA4503, or a salt thereof.

In some embodiments, the compound, or salt thereof, is pridopidine, ANAVEX2-73, SIRA, T-817MA, CT1812, roluperidone (MIN-101), or salt thereof. In some embodiments, the compound, or salt thereof, is pridopidine, or salt thereof. In some embodiments, the compound, or salt thereof, is ANAVEX2-73, or salt thereof. In some embodiments, the compound, or salt thereof, is S1 RA, or salt thereof. In some embodiments, the compound, or salt thereof, is T-817MA, or salt thereof. In some embodiments, the compound, or salt thereof, is CT1812, or salt thereof. In some embodiments, the compound, or salt thereof, is roluperidone (MIN-101), or salt thereof.

In some embodiments, the compound, or salt thereof, is diphenhydramine, or salt thereof.

In some embodiments, a method provided herein further comprises administration of an additional therapeutic agent. In some embodiments, a method provided herein further comprises coadministration of an additional therapeutic agent. In some embodiments, the additional therapeutic agent is an antiviral agent, an immunomodulatory agent, an immunosuppressant, an anti-inflammatory agent, an antibody, or a protein. In some embodiments, the additional therapeutic agent is an antiviral agent, an immunomodulatory agent, an immunosuppressant, an anti-inflammatory agent, or an antibody. In some embodiments, the additional therapeutic agent is an antiviral agent. In some embodiments, the additional therapeutic agent is an immunomodulatory agent. In some embodiments, the additional therapeutic agent is an immunosuppressant. In some embodiments, the additional therapeutic agent is an anti-inflammatory agent. In some embodiments, the additional therapeutic agent is an antibody. In some embodiments, the additional therapeutic agent is a protein. In some embodiments, the immunosuppressant is an antihistamine. In some embodiments, the antihistamine is diphenhydramine. In some embodiments, the additional therapeutic agent is diphenhydramine. In some embodiments, the protein is lactoferrin. In some embodiments, the additional therapeutic agent is lactoferrin.

In some embodiments, a method provided herein comprises coadministration of lactoferrin. In some embodiments, a method provided herein comprises coadministration of lactoferrin and diphenhydramine.

In some embodiments, a method provided herein comprises coadministration of about 1-1,000 μg/mL of lactoferrin. In some embodiments, a method provided herein comprises coadministration of about 50-1,000 μg/mL of lactoferrin. In some embodiments, a method provided herein comprises coadministration of about 50-500 μg/mL of lactoferrin. In some embodiments, a method provided herein comprises coadministration of about 100 μg/mL of lactoferrin. In some embodiments, a method provided herein comprises coadministration of about 200 μg/mL of lactoferrin. In some embodiments, a method provided herein comprises coadministration of about 300 μg/mL of lactoferrin. In some embodiments, a method provided herein comprises coadministration of about 400 μg/mL of lactoferrin. In some embodiments, a method provided herein comprises coadministration of about 500 μg/mL of lactoferrin. In some embodiments, a method provided herein comprises coadministration of about 600 μg/mL of lactoferrin. In some embodiments, a method provided herein comprises coadministration of about 700 μg/mL of lactoferrin. In some embodiments, a method provided herein comprises coadministration of about 800 μg/mL of lactoferrin. In some embodiments, a method provided herein comprises coadministration of about 900 μg/mL of lactoferrin. In some embodiments, a method provided herein comprises coadministration of about 1000 μg/mL of lactoferrin.

In some embodiments, coadministration of an additional therapeutic agent reduces SARS-CoV-2 induced cytotoxicity. In some embodiments, coadministration of lactoferrin reduces SARS-CoV-2 induced cytotoxicity. In some embodiments, coadministration of diphenhydramine reduces SARS-CoV-2 induced cytotoxicity.

In some embodiments, coadministration of an additional therapeutic agent decreases the EC₅₀ of the compound. In some embodiments, coadministration of lactoferrin decreases the EC₅₀ of the compound. In some embodiments, coadministration of diphenhydramine decreases the EC₅₀ of the compound. In some embodiments, coadministration of and additional therapeutic agent decreases the EC₅₀ of the compound by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97.5%, at least 99%, or at least 99.9%. In some embodiments, coadministration of and additional therapeutic agent decreases the EC₅₀ of the compound by at least 5%. In some embodiments, coadministration of and additional therapeutic agent decreases the EC₅₀ of the compound by at least 10%. In some embodiments, coadministration of and additional therapeutic agent decreases the EC₅₀ of the compound by at least 15%. In some embodiments, coadministration of and additional therapeutic agent decreases the EC₅₀ of the compound by at least 20%. In some embodiments, coadministration of and additional therapeutic agent decreases the EC₅₀ of the compound by at least 25%. In some embodiments, coadministration of and additional therapeutic agent decreases the EC₅₀ of the compound by at least 30%. In some embodiments, coadministration of and additional therapeutic agent decreases the EC₅₀ of the compound by at least 35%. In some embodiments, coadministration of and additional therapeutic agent decreases the EC₅₀ of the compound by at least 40%. In some embodiments, coadministration of and additional therapeutic agent decreases the EC₅₀ of the compound by at least 45%. In some embodiments, coadministration of and additional therapeutic agent decreases the EC₅₀ of the compound by at least 50%. In some embodiments, coadministration of and additional therapeutic agent decreases the EC₅₀ of the compound by at least 55%. In some embodiments, coadministration of and additional therapeutic agent decreases the EC₅₀ of the compound by at least 60%. In some embodiments, coadministration of and additional therapeutic agent decreases the EC₅₀ of the compound by at least 65%. In some embodiments, coadministration of and additional therapeutic agent decreases the EC₅₀ of the compound by at least 70%. In some embodiments, coadministration of and additional therapeutic agent decreases the EC₅₀ of the compound by at least 75%.

In some embodiments, coadministration of an additional therapeutic agent further reduces replication of SARS-CoV-2 virus by a greater amount than administration of the compound alone. In some embodiments, coadministration of an additional therapeutic agent further reduces replication of SARS-CoV-2 virus by about 10% more, about 20% more, about 30% more, about 40% more, about 50% more, about 60% more, about 70% more, about 80% more, about 90% more, about 100% more, about 125% more, about 150% more, about 200% more, about 250% more, or about 300% more than administration of the compound alone. In some embodiments, coadministration of an additional therapeutic agent further reduces replication of SARS-CoV-2 virus by about 10% more than administration of the compound alone. In some embodiments, coadministration of an additional therapeutic agent further reduces replication of SARS-CoV-2 virus by about 20% more than administration of the compound alone. In some embodiments, coadministration of an additional therapeutic agent further reduces replication of SARS-CoV-2 virus by about 30% more than administration of the compound alone. In some embodiments, coadministration of an additional therapeutic agent further reduces replication of SARS-CoV-2 virus by about 40% more than administration of the compound alone. In some embodiments, coadministration of an additional therapeutic agent further reduces replication of SARS-CoV-2 virus by about 50% more than administration of the compound alone. In some embodiments, coadministration of an additional therapeutic agent further reduces replication of SARS-CoV-2 virus by about 60% more than administration of the compound alone. In some embodiments, coadministration of an additional therapeutic agent further reduces replication of SARS-CoV-2 virus by about 70% more than administration of the compound alone. In some embodiments, coadministration of an additional therapeutic agent further reduces replication of SARS-CoV-2 virus by about 80% more than administration of the compound alone. In some embodiments, coadministration of an additional therapeutic agent further reduces replication of SARS-CoV-2 virus by about 90% more than administration of the compound alone. In some embodiments, coadministration of an additional therapeutic agent further reduces replication of SARS-CoV-2 virus by about 100% more than administration of the compound alone. In some embodiments, coadministration of an additional therapeutic agent further reduces replication of SARS-CoV-2 virus by about 125% more than administration of the compound alone. In some embodiments, coadministration of an additional therapeutic agent further reduces replication of SARS-CoV-2 virus by about 150% more than administration of the compound alone. In some embodiments, coadministration of an additional therapeutic agent further reduces replication of SARS-CoV-2 virus by about 200% more than administration of the compound alone. In some embodiments, coadministration of an additional therapeutic agent further reduces replication of SARS-CoV-2 virus by about 250% more than administration of the compound alone. In some embodiments, coadministration of an additional therapeutic agent further reduces replication of SARS-CoV-2 virus by about 300% more than administration of the compound alone.

### EXAMPLES

### Example 1

### Introduction

There is a strong need for safe drugs and vaccines to target emerging pathogens such as SARS-CoV-2. Although recent studies identified approved drugs that exhibit antiviral activities against SARS-CoV-2 [1,2], current therapeutic treatment strategies for COVID-19 have limited effectiveness. There are currently no oral medications given emergency use authorization from the Food and Drug Administration to prevent SARS-CoV-2 infection or to treat COVID-19. There is an urgent need to identify safe, economical, orally deliverable approved drugs with activity against SARS-CoV-2 to prevent infection in at-risk populations, and to treat patients experiencing viral disease [3]. Attempts to identify approved drugs with antiviral activity led to the discovery of more than 100 compounds that exhibit direct antiviral activity against SARS-CoV-2 isolates in vitro [2,4-6]. Although the on- and off-target binding mechanisms that mediate anti-SARS-CoV-2 activity are not clear, two classes of molecules were previously found to effectively inhibit virus infectivity: protein biogenesis inhibitors (e.g., zotatifin, ternatin-4, PS3061) and ligands of the sigma-1 and sigma-2 receptors (e.g., haloperidol, clemastine, cloperastine) [7].

Specific antihistamines with off-target antiviral activity may have repurposed utility for prevention and treatment of COVID-19 because of known safety profiles and wide-spread availability. Common antihistamines that exhibit off-target antiviral activity include hydroxyzine, azelastine and diphenhydramine [8]. Mechanisms of action for drugs with direct anti-SARS-CoV-2 activity have important clinical implications in terms of dosing and drug interactions. Defining mechanisms that drive antiviral activity against SARS-CoV-2 will provide rationale for drug combinations targeting distinct antiviral pathways [9]. Drug combinations that target separate antiviral pathways are expected to inhibit drug resistant variants resulting from emerging mutations.

Coronaviruses replicate in a modified compartment derived from the endoplasmic reticulum (ER). The sigma receptor-1 is an ER resident chaperone that normally functions to modulate the ER stress response [10]. Coronavirus infection activates pathways to facilitate adaptation of ER stress to virus proliferation. These pathways are thought to hijack the host cell ER stress response to modulate protein translation, ER protein folding capacity and ER-associated degradation. Targeting the ER stress response could elucidate coronavirus protein-host interactions and provide rationale for new therapeutic approaches to prevention and treatment of COVID-19.

In infected cells, the sigma-1 receptor was shown to link the SARS-CoV-2 replicase/transcriptase complex to the ER membrane by binding directly to nonstructural protein 6 (NSP6) [7]. Although sigma-1 receptor ligands exert antiviral activity against non-coronaviruses and coronaviruses [10], it is not known if agonist or antagonist activities prevent SARS-CoV-2 infection. Understanding binding interactions of antiviral sigma-1 receptor ligands may provide the basis for drug development and optimization.

Although approved drugs that inhibit SARS-CoV-2 in vitro have been shown to bind sigma-2 receptors, the structure and functions of sigma-2 receptors are not well characterized or understood [11]. Sigma-1 and sigma-2 receptors are unrelated in sequence and structure. The sigma-2 receptor is an ER resident membrane protein thought to be involved in hormone, calcium and neuronal signaling [12]. The sigma-2 receptor regulates cholesterol transport and contributes to cholesterol homeostasis [13]. In infected cells, the sigma-2 receptor was shown to bind directly to SARS-CoV-2 ORF9c [7], suggesting that sigma-2 receptor ligands may block host protein:virus protein interactions. Recently, the sigma-1 and -2 ligand PB28 that had sub-nanomolar in vitro SARS-CoV-2 inhibitory effects was found ineffective in vivo (33). The hypothesized cause for poor efficacy was that this compound induced high levels of phospholipidosis in vitro that resulted in virus inhibition that could not be achieved in vivo. Sigma receptor ligands, as a class, should not be discounted because of the poor performance of a single compound. In some embodiments, phospholipidosis induction by sigma ligands does not correlate with inhibition of SARS-CoV-2 viral replication.

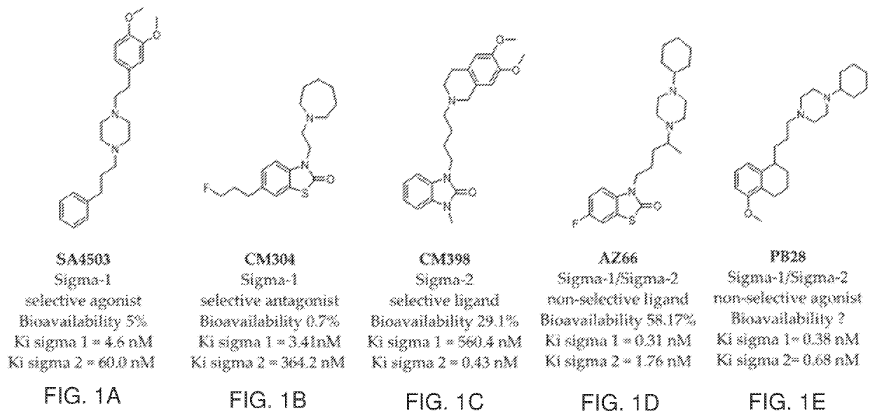

It is clear that multiple sigma receptor ligands exhibit antiviral properties against SARS-CoV-2, but the relative roles of the sigma-1 receptor and sigma-2 receptor agonism and antagonism in modulating antiviral activities are not known. Antiviral activities of highly selective ligands (FIGS. 1A-1E) were measured to define mechanisms driving inhibition of SARS-CoV-2 infection in vitro: a sigma-1 receptor specific agonist (SA4503, cutamesine) [14-16], sigma-1 receptor antagonist (CM304) [17], sigma-2 receptor specific ligand (CM398) [18], and a mixed affinity sigma-1/sigma-2 ligand (AZ66) [19,20]. The benzothiazolone (CM304 and AZ66) and benzimidazolone (CM398) containing compounds were selected for their demonstrated selectivity for sigma receptors against other aminergic transporters or receptors [21]. In addition, the specific compounds were chosen for their differential affinity at the two receptor subtypes, aiming to clarify the involvement of each receptor in the inhibition of SARS-CoV-2 infection in vitro.

### Results

### Sigma Ligands Inhibit SARS-CoV-2-Mediated Cell Death, Intracellular Replication, and Infectivity

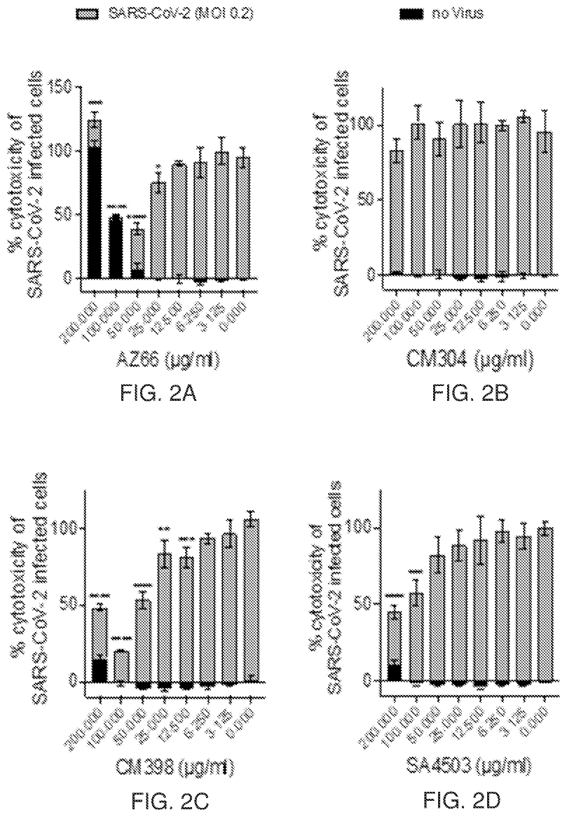

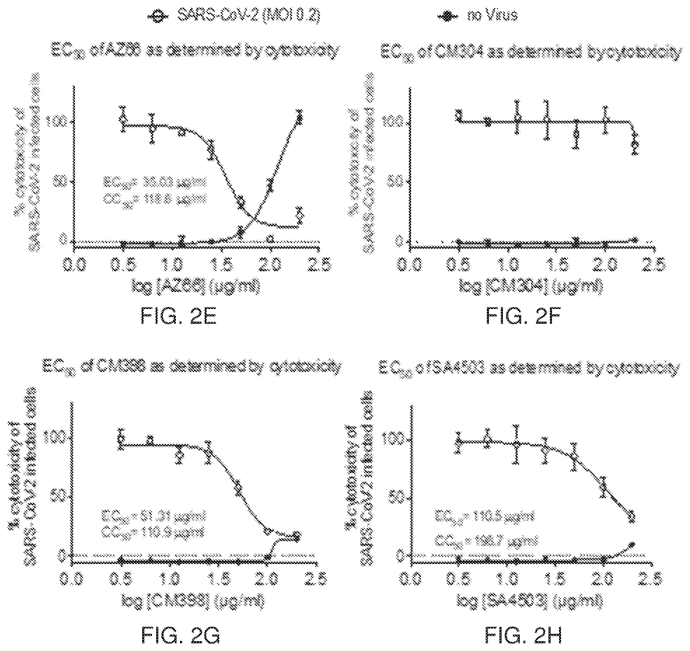

Several assays were used to determine antiviral efficacy of sigma ligands. The African green monkey cell line Vero E6 has been shown to support SARS-CoV-2 infections and was the primary cell line used in the in vitro assays. Initial experiments measuring reduction of SARS-CoV-2 mediated cytotoxicity were carried out at MOI 0.1 and indicated ligands that specifically and non-specifically target sigma receptors had potential for further analysis. With ligand concentrations used in these preliminary studies as a starting point, toxicity and antiviral activities were measured in cytotoxicity assays. In FIGS. 2A-2D, toxicity of ligands alone was observed by the black bars. Superimposed on the black bars are the cytotoxicity values (gray bars) of cells in the presence of the indicated ligand concentrations. The cytotoxic concentration of ligand alone (CC₅₀) and effective concentrations (EC₅₀) of each sigma ligand for inhibition of SARS-CoV-2 induced cytotoxicity were determined by non-linear regression (FIGS. 2E-2H). The specific sigma-1 and sigma-2 receptor ligand AZ66 had the lowest EC₅₀ of the ligands tested at 86.4 μM as measured by cytotoxicity. The sigma-1 receptor antagonist CM304 showed insignificant viral inhibition. However, the sigma-1 receptor agonist SA4503 (cutamesine) had moderate inhibitory activity against SARS-CoV-2-induced cytotoxicity at 299.9 μM. The highly specific sigma 2-receptor ligand CM398 showed significant inhibitory activity and had an EC₅₀ of 129.7 μM. Of the ligands tested in this work, AZ66 had the highest selectivity index (SI; CC₅₀/EC₅₀) ratio as measured by cytotoxicity (>2.82). To compare AZ66 to previously published studies done with remdesivir, a plaque assay was utilized to measure EC₅₀. The reported SI for remdesivir was >4.96 [6] while AZ66 was >19.72 by plaque assay in this work, meaning the gap between SARS-CoV-2 inhibitory concentrations and toxic concentrations is greater for AZ66 than remdesivir (Table 1). The differences in compound efficacy are linked to the ligand binding strength (the lower the Ki) and activity at the sigma receptors (agonism vs. antagonism). The higher EC₅₀ levels from the cytotoxicity assay versus the plaque assay indicate the more sensitive nature of measuring infectious particles. Even low levels of virus can cause cell damage thus requiring higher levels of drug to completely eradicate virus-mediated cytotoxicity.

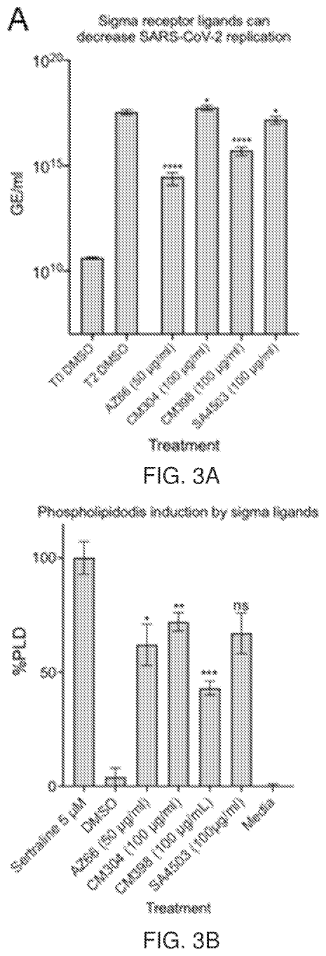

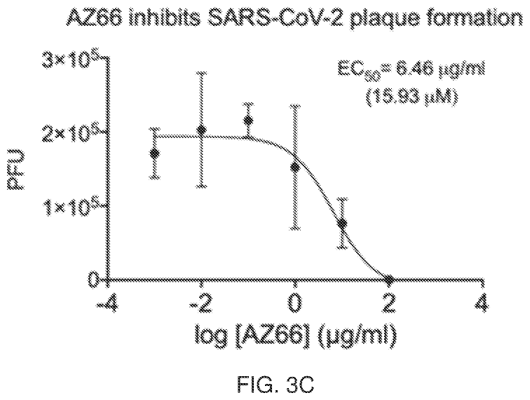

After verifying the ability of ligands to inhibit or reduce cytopathic effects, Vero E6 cells were infected at a low MOI and quantified viral replication through qPCR. Quantitative analysis was used to determine if each ligand inhibited replication of the SARS-CoV-2 genome. The data in FIG. 3A show that in the presence of 50 μg/ml AZ66 viral replication is reduced by 99.9% (3-log). At 100 μg/ml, CM398 is able to reduce viral replication by 97.5% and SA4503 by 56.2%. CM304 was unable to reduce viral replication and RNA levels indicated 61.2% more virus was detected, compared to the 48 hours DMSO control.

To measure whether the inhibitory activity of ligands were correlated with accumulation of phospholipids in cell membranes as a non-specific in vitro SARS-CoV-2 inhibitor, phospholipidosis was measured in H23 human lung epithelial cells (FIG. 3B). CM304 showed the highest mean levels of phospholipidosis followed by SA4503, AZ66, and CM398. CM304 was the least effective SARS-CoV-2 inhibitor tested while having the highest phospholipid accumulation, suggesting a correlation of antiviral activity and phospholipidosis levels was not identified. Since the sigma-1 and -2 ligand AZ66 was the most potent SARS-CoV-2 cytotoxicity and replication inhibitor of those tested, its ability to inhibit plaque formation caused by SARS-CoV-2 (FIG. 3C) was evaluated. The EC₅₀ of AZ66 as determined by plaque reduction assay was 6.46 μg/ml (15.93 μM). In a previous work, the area under the curve (AUC) for AZ66 was 158.22 μg·h/ml following a 20 mg/kg p.o. (oral) dose in rats [22]. The AUC is a measure of tissue exposure to a compound over a period of time. The high AUC for AZ66 indicates in vivo therapeutic levels of AZ66 could be achieved for COVID19 at concentrations that may not induce high levels of phosphorlipidosis in vitro.

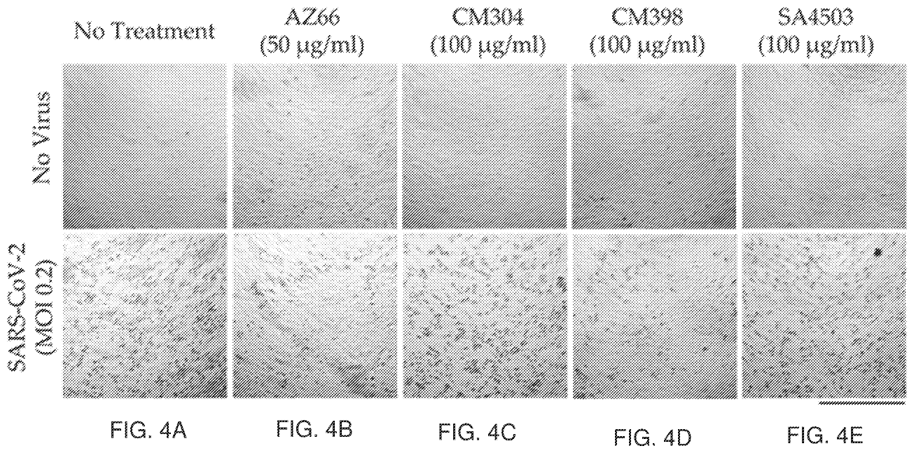

These quantitative data were verified by microscopic evaluation of infected monolayers (FIGS. 4A-4E). The ability of AZ66 and CM398 to greatly reduce cytopathic effects (CPE) caused by SARS-CoV-2 infection is evident as a decrease in cell-rounding and cell death (dark spots, FIGS. 4B and 4D) compared to the no treatment controls (FIG. 4A). CM304 and SA4503 were unable to visibly reduce CPE (FIGS. 4C and 4E). Collectively, these data indicate that antiviral activity against SARS-CoV-2 was driven by agonism of the sigma-1 receptor (e.g., SA4503), and by ligation of the sigma-2 receptor (e.g., CM398).

### Modeled Structural Interactions Between Sigma Receptors and Ligands Provides a Basis for Antiviral Drug Optimization

Since antiviral activity against SARS-CoV-2 was driven by agonism of the sigma-1 receptor (e.g., SA4503, sigma-1 receptor agonist), but not by antagonism of the sigma-1 receptor (CM304, sigma-1 receptor antagonist), ligand interactions were mapped by molecular docking to identify sites on the sigma-1 receptor that could be used as the basis for optimization of antiviral sigma-1 receptor agonists.

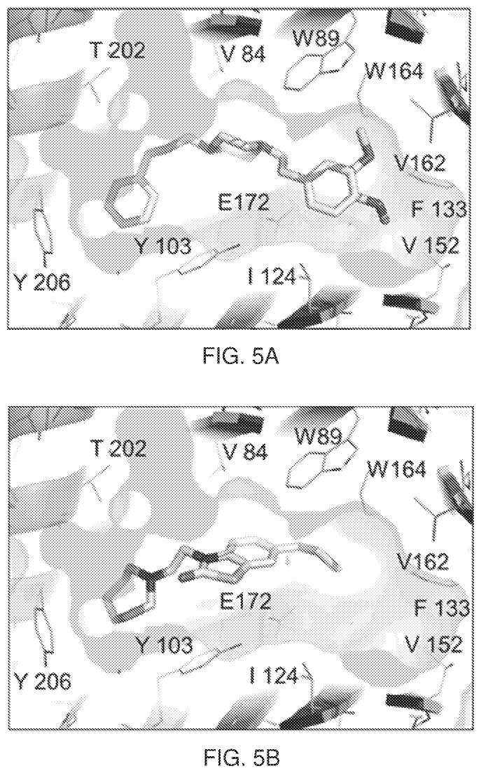

The sigma-1 receptor crystal structure shows the C-terminal domain exhibiting a cupin-like β-barrel with a buried, central ligand-binding site [23]. The ligand binding site of the sigma-1 receptor (PDB 5HK1) was used as the basis for molecular docking simulations to compare active antiviral versus inactive sigma-1 receptor ligands. SA4503, an antiviral sigma-1 receptor agonist, was predicted to form contact with multiple residues in the central ligand-binding site of the sigma-1 receptor: V84, W89, Y103, I124, F133, V152, V162, W164, E172, T202 (FIG. 5A). In contrast, CM304, a highly specific sigma-1 receptor antagonist, but inactive against SARS-CoV-2, formed contact with a subset of residues in the ligand-binding site of the sigma-1 receptor: V84, W89, Y103, E172, T202 (FIG. 5B). These data suggest that sigma-1 receptor binding drugs may be optimized for agonist and antiviral binding activity by forming interactions with specific residues in the sigma-1 receptor ligand-binding site: I124, F133, V152, V162, and W164.

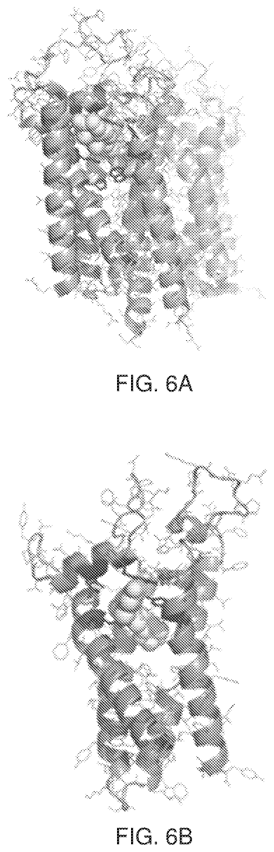

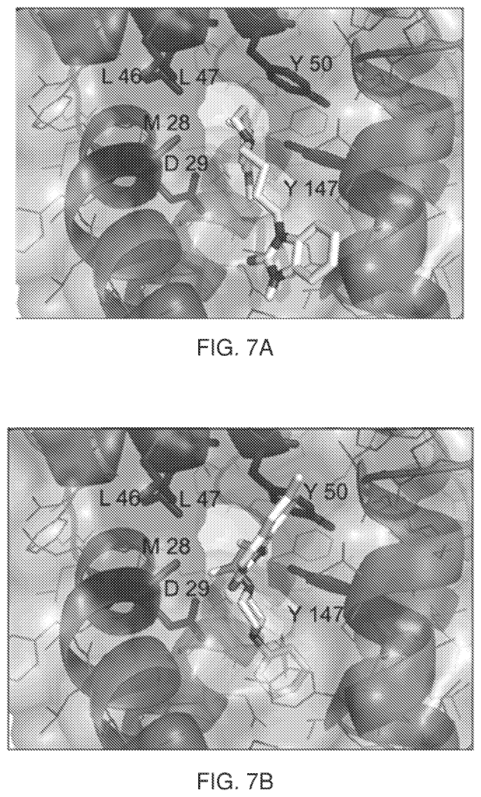

Molecular docking simulations of sigma-2 receptor ligands were performed using a homology model of the human sigma-2 receptor. SWISS-MODEL [24] was used to generate atomic coordinates based on the most similar solved structure, 3-β-hydroxysteroid-Δ8,Δ7-isomerase, known as Emopamil-Binding Protein (EBP) [25], PDB 60HT. EBP, similar to the sigma-2 receptor, is an endoplasmic reticulum membrane protein involved in cholesterol biosynthesis and autophagy. The human sigma-2 receptor, 17.8% identical to EBP, was modeled as a transmembrane protein comprised of α-helices and loop regions that form a putative ligand binding pocket. The structure of EBP was solved complexed to a cholesterol biosynthesis inhibitor UI8666A [26], shown as spheres in FIG. 5A. Sigma-2 receptor ligands that exhibited antiviral activity against SARS-CoV-2 in vitro were docked against the putative ligand binding site of the modeled sigma-2 receptor structure (FIG. 6B). Molecular docking showed that sigma-2 receptor specific ligand CM398, and the sigma-1/sigma-2 receptor ligand AZ66, have the potential to form intermolecular interactions with the ligand-binding site residues (M28, D29, L47, Y50, Y147, shown in grayscale in FIG. 7), equivalent to the ligand-binding site residues of EBP (shown in stick depiction in FIG. 6A). These data provide a structural basis for strategies to optimize antiviral activity against SARS-CoV-2 and selectivity for sigma-1/sigma-2 receptor binding.

Synergistic Antiviral Activity by Combining a Sigma Receptor Ligand with Lactoferrin

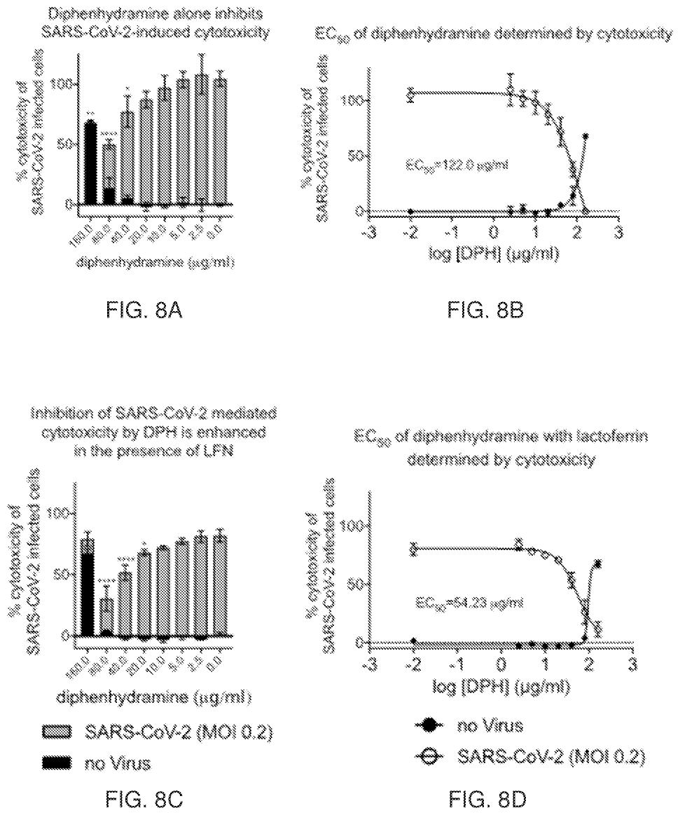

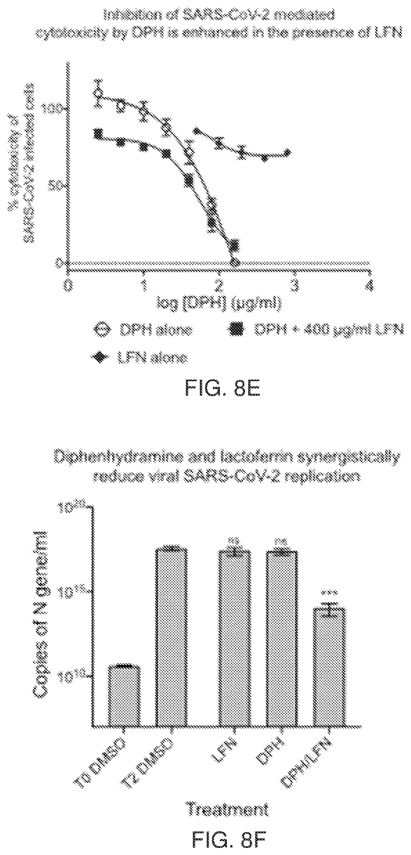

The antihistamine diphenhydramine, with on-target binding to the Histamine-1 receptor, has known off-target effects at the sigma-1 receptor [27]. Diphenhydramine was recently shown to inhibit SARS-CoV-2 infectivity and the calculated EC₅₀ for SARS-CoV-2 by plaque reduction assay was 17.4 μg/ml (59.6 μM). This drug is safe, well-characterized, and widely available and so highly relevant in the search for COVID therapeutics. The ability of diphenhydramine to inhibit SARS-CoV-2 induced cytotoxicity and found an EC₅₀ of 122.0 μg/ml (418 μM; FIGS. 8A and B), about 7 times higher than that found in the plaque reduction assay, similar to the findings with AZ66. It was hypothesized that diphenhydramine could be combined with structurally distinct antiviral agents (binding other receptors, not sigma) to reduce its EC₅₀ for antiviral activity against SARS-CoV-2.

In the investigations into sigma-binding ligands, including diphenhydramine, the intent was to reduce the EC₅₀ by addition of another safe, and well characterized protein from milk, lactoferrin. The host-iron sequestration protein lactoferrin was reported to exhibit direct antiviral activity against SARS-CoV-2 [28,29], is broadly antimicrobial, and possesses host immunostimulatory properties. Combinations of lactoferrin with diphenhydramine were tested to measure effects on reduction of EC₅₀. Co-administration of 400 μg/ml of lactoferrin with diphenhydramine further reduced SARS-CoV-2 induced cytotoxicity and decreased the EC₅₀ by 55.5% to 54.2 μg/ml (185.7 μM; FIGS. 8C and D). The antiviral enhancement effects of lactoferrin are more apparent at lower, therapeutically relevant concentrations of diphenhydramine (FIG. 8E). Inhibition of viral replication was also investigated by qPCR (FIG. 8F). Lactoferrin (400 μg/ml) was able to decrease N-protein RNA copies by 28.0% 48 hours after infection, compared to DMSO alone controls while 40 μg/ml diphenhydramine alone resulted in 32.2% reduction. When combined, they inhibited 99.97% of N-protein RNA copies, a 3-log reduction that was highly significant. These data demonstrate that combinations of two over-the-counter compounds, with well characterized safety profiles, have synergistic effects on inhibition of SARS-CoV-2.

### Sigma Ligands Inhibit Infectious Particle Production in Human Lung Cells

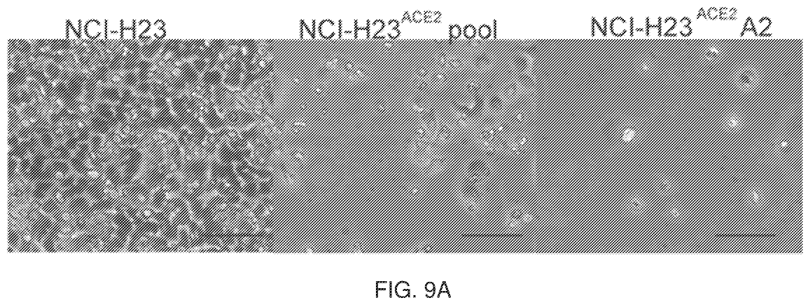

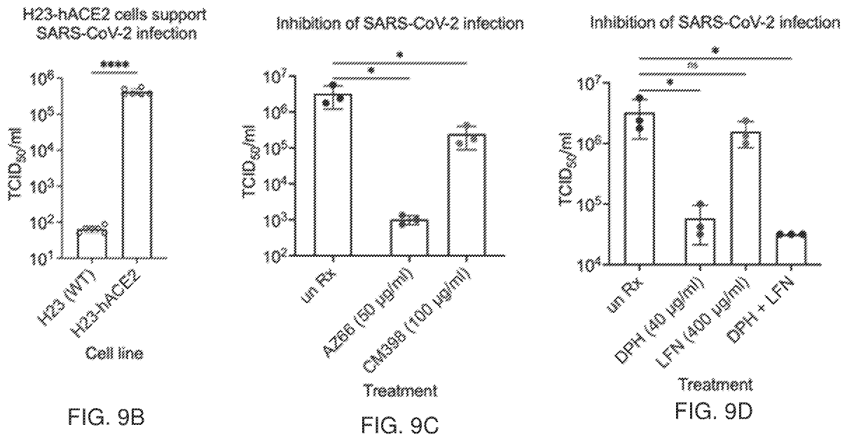

Lastly, inhibition of SARS-CoV-2 infection by compounds shown efficacious in Vero E6 cells was determined in human lung epithelial cells. A new lung cell line susceptible to SARS-CoV-2 infection, H23-ACE2, was generated by lentivirus transduction to introduce the human ACE2 gene. Single clone isolation of the H23-ACE2 transduced cell pool resulted in several healthy clones, including clone A2. Successful ACE2 expression was functionally indicated by increased cytopathic effect upon SARS-CoV-2 infection of an H23-ACE2 cell pool and an isolated cell clone H23-ACE2 clone A2 but not the parental H23 cell line (FIG. 9A). ACE2 surface expression was confirmed by flow cytometry as a peak shift to the right on the X-axis towards for H23-ACE2 cell pool and H23-ACE2 clone A2 compared to the untransduced parent H23 cell line and Vero E6 cells. H23-ACE2 clone A2 was used for further experiments. SARS-CoV-2 was used to infect the human lung epithelial cell line H23 at an MOI of 0.01. This cell line is unable to support SARS-CoV-2 infection without heterologous expression of the ACE-2 receptor [30] and the experiments confirmed the essentiality of hACE2 for infection (FIGS. 9A and 9B). TCID₅₀s were performed to measure infectious particles released during infection in the presence of AZ66, CM398, diphenhydramine, lactoferrin and diphenhydramine+lactoferrin. The mixed affinity sigma-1/sigma-2 receptor ligand AZ66 was able to decrease SARS-CoV-2 concentrations by ˜3-log at 48 hpi compared to mock treated-infected H23-ACE2 cells (FIG. 9C). Cells were originally infected at an MOI of 0.01 which is equivalent to 1.5×10³ virus, so the data show AZ66 completely blocks production of infectious virus particles in these experiments. The sigma-2 receptor specific ligand CM398 was able to reduce SARS-CoV-2 concentrations by ˜1-log. Diphenhydramine effectively reduced SARS-CoV-2 concentrations by ˜2-log while lactoferrin was ineffective (FIG. 9D). The combination of diphenhydramine+lactoferrin showed a combined ability to reduce SARS-CoV-2 replication by half that observed for diphenhdydramine alone. The data from the more physiologically relevant human lung cell lines demonstrate the potential for sigma receptor ligands and drugs with off-target effects on sigma receptors to inhibit SARS-CoV-2 replication.

### Discussion and Conclusions

SARS-CoV-2, the causative virus of COVID-19 pandemic, belongs to a family of positive-sense single-stranded RNA (+ssRNA) coronaviruses (CoVs) that also cause illnesses ranging from common colds to severe diseases such as Middle East respiratory syndrome (MERS). There are 7 CoVs known to infect people: 229E, NL63, OC43, HKU1, MERS-CoV, and SARS-CoV that emerged in 2003 [3]. CoV infection is known to activate pathways that facilitate adaptation of ER stress for viral replication [31]. CoVs utilize host cell ER stress responses to modulate protein translation, ER protein folding capacity, ER-associated degradation (ERAD) including autophagy, and apoptotic cell death [32,33]. It has been proposed that modulation of CoV induced ER stress responses may provide the rationale for new approaches to antiviral drug therapy.

Sigma receptors act as modulators of ER stress, functioning as ligand operated membrane bound chaperones at the ER-mitochondrial contact (mitochondrion-associated ER membrane) [34]. Sigma-1 receptor ligands have been shown to exert antiviral activity against CoVs and non-CoVs, including Ebola, HCV, SARS-CoV, SARS-CoV-2, DENV, MERS-CoV, FLUAV (H5N1), HCV, HIV and HSV-1 [10]. Sigma receptors were implicated as targets for antiviral drugs by mapping interactions between human proteins and 26 (of 29) SARS-CoV-2 proteins, and subsequent screening of approved drugs [7]. Two sets of pharmacological agents effectively inhibited SARS-CoV-2 infectivity in Vero E6 cells: inhibitors of mRNA translation and predicted regulators of the sigma-1 and sigma-2 receptors. Non-selective sigma-1 receptor ligands, including the antihistamines clemastine and cloperastine, exhibited activity against SARS-CoV-2 in vitro. PB28 a sigma-1 and sigma-2 receptor ligand was highly efficacious in vitro [7] but was toxic in vivo so the search for effective ligands was continued in this work [35].

Mechanisms that drive anti-SARS-CoV-2 activity by sigma receptors are not well characterized. It is not understood if both sigma-1 or sigma-2 receptors are involved in antiviral activity, or if agonism, or antagonism of individual receptors mediate antiviral activity. A significant limitation to addressing the role of sigma receptors in SARS-CoV-2 inhibition (of entry, replication or infectious virus assembly/release) is the paucity of structural information available for the sigma-2 receptor, and absence of well characterized agonists and antagonists. Identification of ligands that exert antiviral activity by specific sigma receptor binding may provide the basis for use of existing drugs (repurposed) and for development of new drugs optimized for activity against CoVs.

Combining a sigma receptor ligand with antiviral drugs that bind distinct targets may provide additive or synergistic antiviral effects and decrease the likelihood of SARS-CoV-2 resistance to a single drug. Drug combinations are recommended for antiviral therapy of hepatitis C (e.g., combination of alpha interferon, simeprevir and ribavirin), and HIV [36]. Combinations of drugs that bind host and/or viral proteins have the potential to lessen the severity of COVID-19 by inhibiting virus replication and reducing symptoms. Administration of antiviral drug combinations to SARS-CoV-2 positive patients could determine hospitalization versus home-based care.

Data suggests that specific drugs that bind SARS-CoV-2, or interacting host proteins, also have the potential to prevent COVID-19. For example, hydroxyzine is a first-generation antihistamine that exhibited off-target binding to the SARS-CoV-2 host receptor ACE2 [37] and the sigma-1 receptor. Usage of hydroxyzine (and structurally related antihistamines diphenhydramine and azelastine) was associated with reduced incidence of SARS-CoV-2 positivity in a population of more than 219,000 individuals in California [8]. Hydroxyzine, diphenhydramine and azelastine exhibited direct antiviral activity against SARS-CoV-2 infection of Vero E6 cells in vitro. Since antihistamines act as nasal decongestants and cough suppressants, the on- and off-target binding properties of drugs such as diphenhydramine may have broad utility in prevention and treatment of COVID-19.

In this study, selective sigma receptor ligands (FIG. 1) that drive antiviral activity against SARS-CoV-2 were defined. The dual specificity sigma-1 and sigma-2 receptor ligand AZ66 exhibited antiviral activity against SARS-CoV-2 induced cytotoxicity of Vero E6 cells (FIG. 2A). Since the sigma-1 receptor antagonist CM304 did not inhibit viral cytotoxicity (FIG. 1B), and the sigma-1 receptor agonist SA4503 (cutamesine) exhibited inhibitory activity (FIG. 2D), these data suggest that sigma-1 receptor agonism drives antiviral activity against SARS-CoV-2. Ligation of the sigma-2 receptor may drive antiviral activity independently, since the highly selective sigma-2 receptor ligand CM398 exhibited direct inhibitory activity against SARS-CoV-2 (FIG. 2C). Ligation of both sigma-1 and sigma-2 receptors may elicit higher levels of antiviral activity compared to receptor specific ligands, since AZ66 exhibited the greatest gap between SARS-CoV-2 inhibitory and cellular toxic concentrations (CC₅₀/EC₅₀ ratio, Table 1).

The ability of sigma receptor ligands to exhibit antiviral activity was verified by infecting Vero E6 cells at a low MOI and quantifying viral replication by qPCR (FIG. 3A). The dual sigma receptor ligand AZ66 exhibited the more significant antiviral effects compared to the selective receptor ligands. AZ66 exhibited antiviral activity against SARS-CoV-2 by plaque assay (FIG. 3B). Induction of phospholipidosis by these compounds was measured in human lung epithelial cells (FIG. 3C). All compounds induced phospholipidosis to ˜50% of the positive control. However, a correlation between phospholipidosis and inhibition of virus replication was not identified, and this was consistent with findings from other groups [38]. CM304 was a strong inducer of phospholipidosis yet was ineffective at inhibition of SARS-CoV-2. These data indicate that the antiviral activities of sigma ligands AZ66 and CM398 are driven by specific antiviral inhibitory mechanisms outside of phospholipidosis. These data are consistent with microscopic observation of AZ66 (sigma-1 and sigma-2 receptor ligand) and CM398 (sigma-2 receptor ligand) reducing cell rounding and death caused by SARS-CoV-2 infection (FIG. 4).

Potential interactions between ligands and sigma receptors were mapped to gain insight in intermolecular interactions that promote antiviral activity against SARS-CoV-2. Identification of specific residues in sigma receptors that bind antiviral drugs may provide the basis for drug development strategies to optimize ligand binding.

A crystal structure of the human sigma-1 receptor (PDB 5HK1) was used as the basis for molecular docking of sigma-1 receptor ligands. Comparison of the posed orientations of a sigma-1 receptor agonist (SA4503) with an antagonist (CM304) complexed to the sigma-1 receptor revealed that the agonist (with antiviral activity) formed more intermolecular contacts with the receptor compared to the antagonist (without antiviral activity) (FIG. 5). These data provide the basis for site directed mutagenesis studies to define key ligand binding residues. These data suggest that drugs optimized for sigma-1 receptor agonist and SARS-CoV-2 antiviral activity may be achieved with analogs that form interactions with specific residues in the sigma-1 receptor ligand-binding site: I124, F133, V152, V162, and W164.

Since crystal structures are not available for the human sigma-2 receptor, an atomic homology model was generated based on the most similar solved structure, 3-β-hydroxysteroid-Δ8,Δ7-isomerase, known as Emopamil-Binding Protein (EBP). EBP, a transmembrane protein comprised of α-helices and loop regions that form a ligand binding site, was solved complexed to an inhibitor (FIG. 6A) [25]. Molecular docking was used to simulate ligand binding of sigma-2 receptor specific ligand CM398, and sig-ma-1/sigma-2 receptor ligand AZ66, predicted to form intermolecular interactions with ligand-binding site residues M28, D29. L47, Y50, Y147, FIG. 5B. In addition to the antiviral effects of AZ66, binding of the sigma receptors reduces nociception [17]. The analgesic effect of AZ66 could provide novel treatment of SARS-CoV-2 related pain while inhibiting viral replication. These data provide the basis for mutagenesis and structure-activity-relationship studies to optimize sigma-2 receptor binding and antiviral activity against SARS-CoV-2.

Specific antihistamines exhibit off-target sigma receptor binding activity, and also exhibit antiviral activity against SARS-CoV-2, including clemastine, cloperastine, astemi-zole, hydroxyzine, azelastine and diphenhydramine. Since diphenhydramine is the most commonly used antihistamine exhibiting antiviral activity, it was assessed if antiviral activity could be improved by combining a sigma receptor ligand with lactoferrin, an antiviral agent that binds distinct targets [28,29]. It was demonstrated that co-administration of 400 μg/ml of lactoferrin with diphenhydramine reduced SARS-CoV-2 induced cytotoxicity and decreased the EC₅₀ (FIGS. 8C and 8D). The antiviral enhancement effects of lactoferrin were more apparent at lower, therapeutically relevant concentrations of diphenhydramine (FIG. 8E). Combining lactoferrin with diphenhydramine resulted in synergistic effects on antiviral activity against SARS-CoV-2 (FIG. 8F). Compounds that were effective in Vero E6 were validated in their ability to reduce infectious SARS-CoV-2 production following infection of human lung epithelial cells (FIGS. 9C and 9D). These data suggest that sigma receptor ligands or formulated combinations of over-the-counter products have the potential to inhibit virus infection and/or decrease recovery time from COVID. Lastly, concentrations that inhibited SARS-CoV-2 production were decoupled from phospholipidosis in human lung epithelial cells, suggesting a specific mechanism at the sigma receptors/virus interface. The candidates investigated in this work target sigma receptors that result in selectivity indices higher than remdesivir, a top candidate in large-scale in silico screens that showed efficacy in vitro and in vivo [6].

### Materials and Methods

Sigma Ligands and Other Drugs Used in this Study.

AZ66, CM304, CM398, and SA 4503 (cutemisine) were obtained by synthesis or from commercial sources and diluted in PBS to 2 mg/ml and frozen at −80° C. in aliquots to eliminate freeze thaw cycles. AZ66, CM304, and CM398 were synthesized with purities ≥95% each. SA 4503 (cutamesine) was obtained from MilliporeSigma (St. Lou-is, MO) at >98% purity. Lactoferrin from human milk was obtained from MilliporeSigma at >85% purity and diphenhydramine HCl was purchased from Spectrum Pharmaceuticals at ≥98% purity.

### Virus Culture Methods.

The SARS-CoV-2 strain used in this study was UF-1. It has been described previously and was isolated from a COVID19 patient at UF Health Shands Hospital via nasal swab. Virus experiments were carried out under a University of Florida Institutional Biosafety Committee-approved protocol in a Biosafety Level 3 laboratory at the Emerging Pathogens Institute. The accession number of the previously sequenced strain can be found under the following GenBank accession number: MT295464.1. Vero E6 cells purchased from ATCC were used to propagate virus using standard methods. Vero E6 cells were grown in DMEM+2% FBS+PenStrep. SAEC, H23 and H23-hACE2 cells were grown in RPMI+10% FBS+PenStrep with 4 μg/ml of blasticidin to maintain ACE2 expression if needed. Cell were grown at 37° C. and 5% CO₂ in a humidified incubator. An EVOS XL Core microscope was used to visualize cells in the BSL3.

Quantitation of Virus Replication by qPCR.

SARS-CoV-2 was used to infect Vero E6 monolayers at an MOI of 0.01 in the presence of each treatment in biological and technical triplicate. At 2 days post-infection (dpi), the monolayers were scraped and harvested into viral lysis buffer (buffer AVL) from the QIAamp Viral RNA Kit (QIAGEN). The AVL buffer is a CDC approved method of viral inactivation. Samples were frozen at −80° C. and removed from the BSL3. RNA was purified according to the manufacturer's recommendations. Reverse transcription and cDNA synthesis was accomplished using the iTaq Universal SYBR Green One-Step Kit (BioRad) and primers targeting the nucleocapsid (N) gene of SARS-CoV-2 (NproteinF-GCCTCTTCTCGTTCCTCATCAC, NproteinR-AGCAGCATCACCGCCATTG). qPCR was carried out on a BioRad CFX96. N protein copy levels were calculated using CT values from a standard curve generated using a control plasmid containing the N protein gene and are presented as genome equivalents (GE) (Integrated DNA Technologies).

### Sigma Ligand Cytotoxicity Reduction Assays.

Vero E6 cells were seeded into 96-well CellBind treated plates (Corning) and allowed to attach overnight. Drugs were pre-aliquoted in DMEM+2% FBS. Cells and drug dilutions were transported into the BSL3 laboratory where titered SARS-CoV-2 aliquots were diluted to produce a target MOI of 0.2 PFU/cell in solution at the final indicated drug concentrations. Triplicate monolayers were infected by replacing growth media with 100 μl of the drug/virus suspensions. At 72 hours post infection, supernatants were harvested, and lactate dehydrogenase (LDH) release was assayed using the Cytox 96™ Non-Radioactive Cytotoxicity Assay (Promega). Assays were performed as recommended by the manufacturer to generate a formazan dye. The optical density at 450 nm was measured using a MultiSkan FC plate reader (ThermoFisher). Controls included total LDH release as measured by lysis of all cells, spontaneous release from uninfected cells, and media alone. The toxicity of sigma ligands alone were also determined in parallel to discriminate the amount of SARS-CoV-2-induced cytotoxicity occurring in the presence of a given treatment. After spontaneous and background subtraction, OD₄₅₀ values were transformed to a percent of SARS-CoV-2 infected cells (100%) in the absence of any drug treatment to obtain percent of SARS-CoV-2-induced cytotoxicity. These experiments were carried out twice.

### Plaque Reduction Assay.

Vero E6 cells were plated in 24-well plates with triplicate replicates on different plates. Virus master mix was used to dilute down to 20-200 PFU/ml and aliquoted in separate tubes with drugs at the final indicated drug concentrations. The drug virus mixtures were immediately used to infect Vero E6 monolayers for 1 hour with rocking every 10 min. Monolayers were then overlaid with MEM in 1.5% low-melt agarose containing drugs at the final concentrations indicated. Plaques were counted at 72 hours post infection and used to calculate the apparent reduction in viral concentration compared to the starting volume. Data presented is representative of two independent experiments.

### Generation of ACE-2 Lentivirus Particles

The lentivirus containing ACE2 were generated by co-transfecting psPAX2, pMD2. G, and an ACE expression vector that also contained a blasticidin selection gene EX-U1285-Lv197 (GeneCopoeia). The plasmids were transfected into HEK293T cells using X-tremeGENE 9 (Roche Cat #XTG9-RO) as per the manufacturer's instructions. Media was replaced with DMEM containing 2% (w/v) bovine serum albumin (BSA) 18 hours post trans-fection and then lentiviruses were collected after 24 hours and 48 hours [41].

### ACE2 Transduction of NCI-H23 Cells and Monoclonal Cell Selection

NCI-H23 (aka H23) cells were obtained from ATCC (CRL-5800) and ACE2 lentiviruses were filtered through a 0.45 μm filter and used to transduce H23 cells using reverse transduction. Briefly, filtered virus particles are added to the H23 cell suspension with RPMI 1640 (Gibco Cat #1185093) media supplemented with 10% FBS and 8 μg/ml polybrene (Sigma Cat #TR-1003-G), 72 hours post transduction, media was changed to RPMI 1640 supplemented with 10% FBS and 4 μg/ml blasticidin S hydrochloride-(Gibco Cat #R21001). Cells were expanded in increasingly larger cell culture plates and ACE2 expression was confirmed by infecting with SARS-CoV-2 (strain Canada/ON/VIDO-01/2020) and flow cytometry. Single clone isolation from the H23-ACE2 cell pool was carried out by the array dilution method in 96-well plates. Single clones were collected 2-3 weeks after seeding and expanded in increasingly larger cell culture plates. After successful isolation, cells were maintained with complete media containing 2 μg/ml blasticidin.

### Analysis of Cell Surface ACE2 by Flow Cytometry

Healthy cells were detached from the monolayer using 0.5 mM EDTA in PBS and centrifuged at 1500 rpm for 3 minutes. The cell pellet was stained for 1 hour at 4° C. with primary ACE2 antibody (R&D systems Cat #AF933, used at a concentration of 0.25 μg/106 cells). The cells were then washed twice with flow wash buffer (2% FBS in PBS) and stained with secondary Goat IgG APC conjugated antibody (R&D systems Cat #F0108, at recommended volume of 10 μl/106 cells), 1000× live-dead viability stain (Invitrogen Cat #L34958) and fixed with 2% PFA (diluted in flow wash buffer). The cells were analyzed using a Beck-man CytoFLEX Flow Cytometer and the CytoExpert software.

### TCID₅₀ Assays in H23 Cells

H23 or H23-hACE2 cells were seeded at 1.5×105 cells in Corning CellBIND 24-well plates and allowed to attach overnight. The next day, SARS-CoV-2 was used to infect the cells at an MOI of 0.01 in the presence of mock treatment (PBS), AZ66 (50 μg/ml), CM398 (100 μg/ml), diphenhydramine (40 μg/ml), lactoferrin from human milk (400 μg/ml), or a combination of diphenhydramine (40 μg/ml) and lactoferrin (400 μg/ml). The TCID₅₀s were performed by diluting 48 hours supernatant from the H23 infections across 8 columns of Vero E6 cells in three independent experiments. Five days later the TCID plates were observed by microscopy for CPE. TCID₅₀/ml in the original H23 infection culture supernatant were calculated by the method of Spearman-Karber. The TCID experiments were carried out in technical triplicate as described above with individual TCID₅₀/ml values and their average and standard deviation shown.

### Inhibitory Concentration and Effective Concentration Calculations.

Regardless of whether the assay was cytotoxicity or plaque reduction, CC₅₀ values and EC₅₀ values were calculated using the GraphPad Prism 9 software nonlinear regression module.

TABLE 1
Cytotoxicity and plaque reduction values of sigma receptor ligands.

| | Cytotoxicity | | | | Plaque reduction | |
|---|---|---|---|---|---|---|
| | CC₅₀ | | EC₅₀ | | EC₅₀ | |
| | μg/ml | R² | μg/ml | R² | μg/ml | R² |
| AZ66 | 127.6 | 0.9957 | 45.14 | 0.8874 | 6.47 | 0.7369 |
| | (109.8- | | (36.91- | | (1.27- | |
| | 173.7) | | 55.21) | | 32.86) | |
| CM398 | 110.9 | 0.9618 | 51.31 | 0.9629 | | |
| | (ND) | | (43.78- | | | |
| | | | 61.13) | | | |
| SA4503 | 198.7 | 0.9401 | 110.5 | 0.8789 | | |
| | (ND) | | (39.2- | | | |
| | | | 311.3) | | | |

### Molecular Docking of Sigma Receptor Ligands

Sigma receptor ligands were docked individually using AutoDock Vina 42 into the ligand binding site of the sigma-1 receptor (PDB 5HK1). The SMILES string of each compound was translated into 3 dimensional coordinates using the NCI/CADD translator (cactus.nci.nih.gov/translate/). AutoDock Tools [42] assigned hydrogen atoms and calculated atom charges for AutoDock Vina. Atomic coordinates for ligand PD144418 and solvent molecules were extracted from the sigma 1 receptor structure and each compound was docked to the ligand binding site using AutoDock Vina. The top 9 scoring orientations were evaluated by visual inspection with the highest scoring poses reported. PyMol (pymol.org/2/) was used to measure interatomic distances and identify sigma-1 receptor residues implicated in ligand binding. An atomic model of the human sigma-2 receptor was generated using SWISS-MODEL based on the most similar solved structure, 3-β-hydroxysteroid-Δ8,Δ7-isomerase, also known as Emopamil-Binding Protein (EBP), PDB 60HT. EBP was solved complexed to an inhibitor, UI8666A, and provided the basis for a putative sigma-2 receptor binding site. AutoDock Vina was used for molecular docking simulations of sigma-2 receptor ligands to the modeled human sigma-2 receptor. Figures generated with PyMol.

### Phospholipidosis Assay

Phospholipidosis assay was performed with the acCELLerate GmbH (Hamburg, Germany) InstaCELL® Phospholipidosis assay kit per the manufacturer's protocol using H23 cells. The cells were seeded in a 96 well plate at a seeding density of 1.5×10⁴ cells per well and allowed to grow in an incubator at 37° C., 5% CO₂. After 24 hours, cells were treated with 50 μg/ml of AZ66 and 100 for each of CM304, CM398 or SA4503, along with positive (sertraline, 5 μM) and vehicle (DMSO, 0.5%) controls, and incubated for 48 hours. At the end of incubation, cells were washed with PBS buffer and stained with PLD staining solution (LysoID/Hoechst phospholipids staining solution) for 30 minutes. Thereafter the cells were washed with PBS buffer, and fluorescence was measured using a microplate reader (SpectraMax iD3, Molecular Devices LLC) at 540 nm excitation/680 nm emission and normalized against fluorescence at 340 nm excitation/480 nm emission.

### Example 2

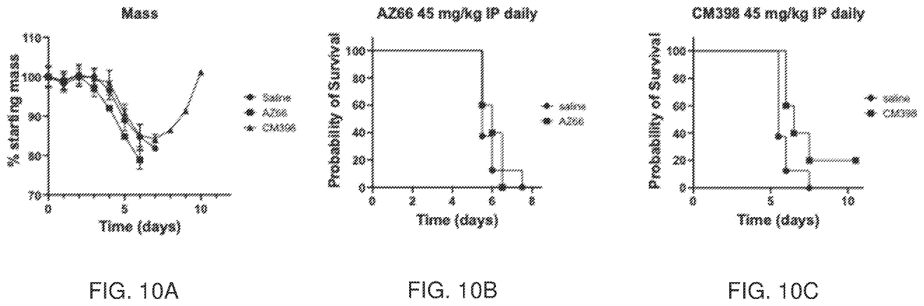

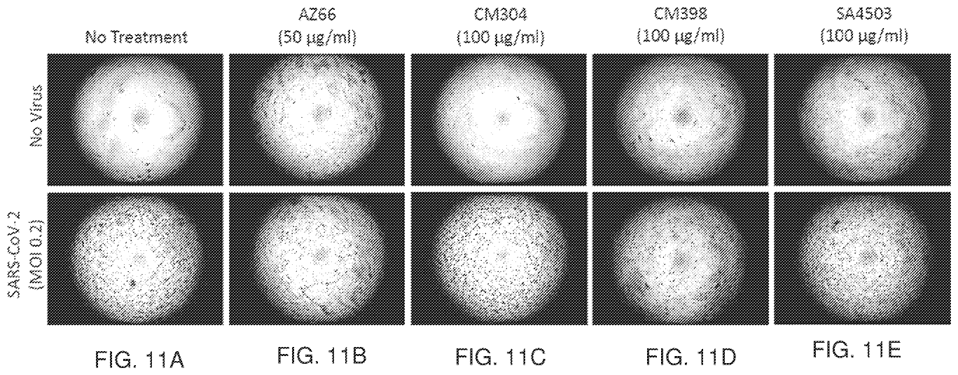

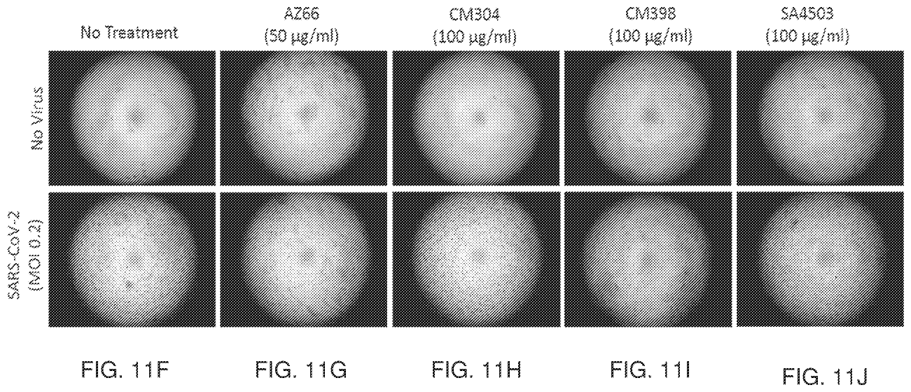

6-8 week old K18-hACE2 mice were ketamine xylazine anesthetized and infected with 2.66×10\hat{ }4 PFU of SARS-CoV-2 by the intranasal route, 24 hours after infection, a group of 8 mice was administered daily intraperitoneal injections of saline, 24 hours after infection, groups of 5 mice were administered daily intraperitoneal injections of either 45 mg/kg of AZ66 or 45 mg/kg of CM398. Mice were weighed each day and survival was monitored (FIGS. 10A-10C). While not statistically significant, median survival increased by one day for mice that received CM398 (FIG. 10C, 5.5 days for saline and 6.5 days for CM398). One mouse in the CM398 group survived and regained all weight lost, which is considered a full recovery. Each of AZ66 and CM398 delayed weight loss associated with COVID-19.

### Example 3

6-8 week old K18-hACE2 mice are ketamine xylazine anesthetized and infected with 2.66×10\hat{ }4 PFU of SARS-CoV-2 by the intranasal route, 6 hours after infection, groups of 5 mice are administered daily intraperitoneal injections of either saline; 30 mg/kg, 60 mg/kg, 75 mg/kg, 90 mg/kg, or 120 mg/kg of AZ66; or 30 mg/kg, 60 mg/kg 75 mg/kg, 90 mg/kg, or 120 mg/kg of CM398. Mice are weighed each day and survival is monitored.

### Example 4

6-8 week old K18-hACE2 mice are pre-treated with intraperitoneal injections of either saline; 30 mg/kg, 45 mg/kg, 60 mg/kg, 75 mg/kg, 90 mg/kg, or 120 mg/kg of AZ66; or 30 mg/kg, 45 mg/kg, 60 mg/kg, 75 mg/kg, 90 mg/kg, or 120 mg/kg of CM398. Mice are ketamine xylazine anesthetized and infected with 2.66×10\hat{ }4 PFU of SARS-CoV-2 by the intranasal route, 6 hours or 24 hours after infection, groups of 5 mice are administered daily intraperitoneal injections of either saline; 30 mg/kg, 45 mg/kg, 60 mg/kg, 75 mg/kg, 90 mg/kg, or 120 mg/kg of AZ66; or 30 mg/kg, 45 mg/kg, 60 mg/kg, 75 mg/kg, 90 mg/kg, or 120 mg/kg of CM398. Mice are weighed each day and survival monitored.

### Erample 5

6-8 week old K18-hACE2 mice are ketamine xylazine anesthetized and infected with 2.66×10\hat{ }4 PFU of SARS-CoV-2 by the intranasal route. Beginning either 6 hours or 24 hours after infection, groups of 5 mice are administered daily intraperitoneal injections of either saline; 30 mg/kg, 45 mg/kg, 60 mg/kg, 75 mg/kg, 90 mg/kg, or 120 mg/kg of AZ66; or 30 mg/kg, 45 mg/kg, 60 mg/kg, 75 mg/kg, 90 mg/kg, or 120 mg/kg of CM398. Mice are weighed each day. Mice are euthanized at day 3 post-infection, and the viral load in the lungs measured to evaluate the ability of AZ66 or CM398 to reduce viral replication. TCID₅₀ is used for enumerating infectious particles in samples. The lungs are homogenized in PBS, then submitted to TCID₅₀. TCID₅₀ procedures are described in Example 1 and in Lei et al. On the Calculation of TCID₅₀ for Quantitation of Virus Infectivity. Virol, Sin. 2021, 36, 141-144 [43].

The SARS-CoV-2 mouse infection model used in Examples 2-5 is described in Winkler, et al. SARS-CoV-2 infection of human ACE2-transgenic mice causes severe lung inflammation and impaired function. Nature Immunology 2021, 21, 1327-1335 [44] and by the Jackson Laboratory (Strain #034860 I Common Name: K18-hACE2).

### REFERENCES

1. Jeon, S.: Ko, M.; Lee, J.; Choi, I.; Byun, S. Y.; Park, S.; Shum, D.; Kim, S. Identification of Antiviral Drug Candidates against SARS-CoV-2 from FDA-Approved Drugs. Antimicrob. Agents Chemother. 2020, 64, e00819-20, doi:e00819-20 [pii].
2. Joshi, S.: Joshi, M.; Degani, M. S. Tackling SARS-CoV-2: Proposed targets and repurposed drugs. Futur. Med. Chem. 2020, 12, 1579-1601, doi:10.4155/fmc-2020-0147 [doi].
3. Zhou, Y.; Hou, Y.; Shen, J.; Huang, Y.; Martin, W.; Cheng, F. Network-based drug repurposing for novel coronavirus 2019-nCoV/SARS-CoV-2. Cell Discov. 2020, 6, 1-18, doi:10.1038/s41421-020-0153-3 [doi].
4. Poduri, R.; Joshi, G.; Jagadeesh, G. Drugs targeting various stages of the SARS-CoV-2 life cycle: Exploring promising drugs for the treatment of covid-19. Cell Signal. 2020, 74, 109721, doi:S0898-6568(20)30198-4 [pii].
5. Riva, L.; Yuan, S.: Yin. X.; Martin-Sancho, L.; Matsunaga, N.; Pache, L.: Burgstaller-Muehlbacher, S.; De Jesus, P. D.; Teriete, P.: Hull, M. V.; et al. Discovery of SARS-CoV-2 antiviral drugs through large-scale compound repurposing. Nature 2020, 586, 113-119, doi:10.1038/s41586-020-2577-1.
6. Jang, W. D.; Jeon, S.: Kim, S.: Lee, S. Y. Drugs repurposed for COVID-19 by virtual screening of 6,218 drugs and cell-based assay. Proc. Natl. Acad. Sci. USA 2021, 118(30), doi:10.1073/pnas.2024302118 [pii].
7. Gordon, D. E.; Jang, G. M.; Bouhaddou, M.: Xu, J.; Obernier, K.; White, K. M.; O'Meara, M. J.: Rezelj, V. V.; Guo, J. Z.; Swaney, D. L.: et al. A SARS-CoV-2 protein interaction map reveals targets for drug repurposing. Nature 2020, 583, 459-468, doi:10.1038/s41586-020-2286-9.
8. Reznikov, L. R.: Norris, M. H.; Vashisht, R.; Bluhm, A. P.; Li, D.: Liao, Y.-S. J.: Brown, A.; Butte, A. J.; Ostrov, D. A. Identification of antiviral antihistamines for COVID-19 repurposing. Biochem. Biophys. Res. Commun. 2021, 538, 173-179, doi:10.1016/j.bbrc.2020.11.095.
9. Xiu, S.: Dick, A.: Ju, H.; Mirzaie, S.; Abdi, F.; Cocklin, S.; Zhan, P.: Liu, X. Inhibitors of SARS-CoV-2 Entry: Current and Future Opportunities. J. Med. Chem. 2020, 63, 12256-12274, doi:10.1021/acs.jmedchem.0c00502.
10. Vela, J. M. Repurposing Sigma-1 Receptor Ligands for COVID-19 Therapy? Front. Pharmacol. 2020, 11, 582310, doi:10.3389/fphar.2020.582310.
11. Abate, C.; Niso, M.: Berardi, F. Sigma-2 receptor: past, present and perspectives on multiple therapeutic exploitations. Futur. Med. Chem. 2018, 10, 1997-2018, doi:10.4155/fmc-2018-0072.
12. Zeng, C.: Riad, A.; Mach, R. H. The Biological Function of Sigma-2 Receptor/TMEM97 and Its Utility in PET Imaging Studies in Cancer. Cancers 2020, 12, 1877, doi:10.3390/cancers12071877.
13. Yang, K.; Zeng, C.; Wang, C.; Sun, M.; Yin, D.; Sun, T. Sigma-2 Receptor-A Potential Target for Cancer/Alzheimer's Disease Treatment via Its Regulation of Cholesterol Homeostasis. Molecules 2020, 25, 5439, doi:10.3390/molecules25225439.
14. Li, D.; Zhang, S.-Z.; Yao, Y.-H.; Xiang, Y.; Ma, X.-Y.; Wei, X.-L.; Yan, H.-T.; Liu, X.-Y. Sigma-1 receptor agonist increases axon outgrowth of hippocampal neurons via voltage-gated calcium ions channels. CNS Neurosci. Ther. 2017, 23, 930-939, doi:10.1111/cns.12768.
15. Kawamura, K.: Ishiwata, K.; Tajima, H.; Ishii, S.-I.; Matsuno, K.: Homma, Y.; Senda, M. In vivo evaluation of [11C]SA4503 as a PET ligand for mapping CNS sigmal receptors. Nucl. Med. Biol. 2000, 27, 255-261, doi:10.1016/s0969-8051(00)00081-0.
16. Matsuno, K.; Nakazawa, M.; Okamoto, K.; Kawashima, Y.; Mita, S. Binding properties of SA4503, a novel and selective sigma 1 receptor agonist. Eur J. Pharmacol. 1996, 306, 271-279, doi:0014-2999(96)00201-4 [pii].
17. Cirino, T. J.; Eans, S. O.; Medina, J. M.: Wilson, L. L.; Mottinelli, M.; Intagliata, S.; McCurdy, C. R.; McLaughlin, J. P. Characterization of Sigma 1 Receptor Antagonist CM-304 and Its Analog, AZ-66: Novel Therapeutics Against Allodynia and Induced Pain. Front. Pharmacol. 2019, 10, 678, doi:10.3389/fphar.2019.00678.
18. Intagliata, S.; Sharma, A.; King, T.; Mesangeau, C.; Seminerio, M.; Chin, F. T.: Wilson, L. L.; Matsumoto, R. R.; McLaughlin, J. P.; Avery, B. A.; et al. Discovery of a Highly Selective Sigma-2 Receptor Ligand, 1-(4-(6,7-Dimethoxy-3,4-dihydroisoquinolin-2(1H)-yl)butyl)-3-methyl-1H-benzo[d]imidazol-2(3H)-one (CM398), with Drug-Like Properties and Antinociceptive Effects In Vivo. AAPS J. 2020, 22, 1-11. doi:10.1208/s12248-020-0472-x.
19. Jamalapuram, S.; Vuppala, P. K.; Abdelazeem, A. H.; McCurdy, C. R.; Avery, B. A. Ultra-performance liquid chromatography tandem mass spectrometry method for the determination of AZ66, a sigma receptor ligand, in rat plasma and its application to in vivo pharmacokinetics. Biomed. Chromatogr. 2013, 27, 1034-40, doi:10.1002/bmc.2901.
20. Xu, Y.-T.; Kaushal, N.; Shaikh. J.; Wilson, L. L.; Mésangeau, C.; McCurdy, C. R.; Matsumoto, R. R. A Novel Substituted Piperazine, CM156, Attenuates the Stimulant and Toxic Effects of Cocaine in Mice. J. Pharmacol. Exp. Ther. 2010, 333, 491-500, doi:10.1124/jpet.109.161398.
21. Mésangeau, C.; Narayanan, S.; Green, A. M.; Shaikh. J.; Kaushal, N.; Viard, E.; Xu, Y.-T.; Fishback, J. A.; Poupaert, J. H.; Matsumoto, R. R.; et al. Conversion of a Highly Selective WO 2022/169961 PCT/US2022/0150% Sigma-1 Receptor-Ligand to Sigma-2 Receptor Preferring Ligands with Anticocaine Activity. J. Med. Chem. 2008, 51, 1482-1486, doi:10.1021/jm701357m.
22. Seminerio, M. J.: Robson, M. J.; Abdelazeem, A. H.: Mesangeau, C.; Jamalapuram, S.: Avery, B. A.: McCurdy, C. R.: Matsumoto, R. R. Synthesis and Pharmacological Characterization of a Novel Sigma Receptor Ligand with Improved Metabolic Stability and Antagonistic Effects Against Methamphetamine. AAPS J. 2012, 14, 43-51, doi:10.1208/s12248-011-9311-8.
23. Shi, Y.; Shuai, L.; Wen, Z.; Wang, C.: Yan, Y.; Jiao, Z.; Guo, F.; Fu, Z. F.; Chen, H.; Bu, Z.; et al. The preclinical inhibitor GS441524 in combination with GC376 efficaciously inhibited the proliferation of SARS-CoV-2 in the mouse respiratory tract. Emerg. Microbes Infect. 2021, 10, 481-492, doi:10.1080/22221751.2021.1899770.
24. Arnold, K.: Bordoli, L.; Kopp, J.; Schwede, T. The SWISS-MODEL workspace: a web-based environment for protein structure homology modelling. Bioinformatics 2006, 22, 195-201, doi:10.1093/bioinformatics/bti770.
25. Long, T.; Hassan, A.; Thompson, B. M.: McDonald, J. G.: Wang, J.; Li, X. Structural basis for human sterol isomerase in cholesterol biosynthesis and multidrug recognition. Nat.

Commun. 2019, 10, 1-8, doi:10.1038/s41467-019-10279-w.
26. Hernsndez, J.; Gabrielli, M.: Costa, J.: Uttaro, A. D. Phagocytic and pinocytic uptake of cholesterol in Tetrahymena thermophila impact differently on gene regulation for sterol homeostasis. Sci. Rep. 2021, 11, 1-11, doi:10.1038/s41598-021-88737-z.
27. Basile, A. S.; A Paul, I.: Mirchevich, A.: Kuijpers, G.; De Costa, B. Modulation of (+)-[3H]pentazocine binding to guinea pig cerebellum by divalent cations. Mol. Pharmacol. 1992, 42, 882-889.
28. Mirabelli, C.: Wotring, J. W.; Zhang, C. J.; McCarty, S. M. Fursmidt, R.; Pretto, C. D.; Sexton, J. Z. Morphological cell profiling of SARS-CoV-2 infection identifies drug repurposing candidates forCOVID-19, bioRxiv 2020, 338, e2105815118, doi:2020.05.27.117184 [pii].
29. Hu, Y.; Meng, X.; Zhang, F.; Xiang, Y.; Wang, J. The in vitro antiviral activity of lactoferrin against common human coronaviruses and SARS-CoV-2 is mediated by targeting the heparan sulfate co-receptor. Emerg. Microbes Infect. 2021, 10, 317-330, doi:10.1080/22221751.2021.1888660.
30. Li, F.; Han, M.; Dai, P.; Xu, W.; He, J.; Tao, X.; Wu, Y.; Tong, X.; Xia, X.; Guo, W.; et al. Distinct mechanisms for TMPRSS2 expression explain organ-specific inhibition of SARS-CoV-2 infection by enzalutamide. Nat. Commun. 2021, 12, 1-14, doi:10.1038/s41467-021-21171-x.
31. Sola, I.; Almazán, F.; Zúñiga, S.; Enjuanes, L. Continuous and Discontinuous RNA Synthesis in Coronaviruses. Annu. Rev. Virol. 2015, 2, 265-288, doi:10.1146/annurev-virology-100114-055218.
32. Fung, T. S.; Liu, D. X. Coronavirus infection, ER stress, apoptosis and innate immunity.

Front. Microbiol. 2014, 5, 296, doi:10.3389/fmicb.2014.00296.
33. Fung, T. S.; Huang, M.; Liu, D. X. Coronavirus-induced ER stress response and its involvement in regulation of coronavirus-host interactions. Virus Res. 2014, 194, 110-123, doi:10.1016/j.virusres.2014.09.016.
34. Hayashi, T. The Sigma-1 Receptor in Cellular Stress Signaling. Front. Neurosci. 2019, 13, 733, doi:10.3389/fnins.2019.00733.
35. Tummino, T. A.; Rezelj, V. V.; Fischer, B.; Fischer, A.; O'Meara, M. J.; Monel, B.: Vallet, T.; White, K. M.; Zhang, Z.; Alon, A.; et al. Drug-induced phospholipidosis confounds drug repurposing for SARS-CoV-2. Science 2021, 373, 541-547, doi:10.1126/science.abi4708.
36. Zheng, Y.: Ma, S.; Xiong, Y.; Fan, X. Efficacy and safety of direct acting antiviral regimens for hepatitis C virus and human immunodeficiency virus co-infection: systematic review and network meta-analysis. J. Gastroenterol. Hepatol. 2020, 35, 1477-1487, doi:10.1111/jgh.15051.
37. Kulemina, L. V.; Ostrov, D. A. Prediction of Off-Target Effects on Angiotensin-Converting Enzyme 2. J. Biomol. Screen. 2011, 16, 878-885, doi:10.1177/1087057111413919.
38. Lane, T. R.; Ekins, S. Defending Antiviral Cationic Amphiphilic Drugs That May Cause Drug-Induced Phospholipidosis. J. Chem. Inf. Model. 2021, 61, 4125-4130, doi:10.1021/acs.jcim.Ic00903.
39. Seminerio, M. J.; Robson, M. J.; Abdelazeem, A. H.; Mesangeau, C.; Jamalapuram, S.; Avery, B. A.; McCurdy, C. R.; Matsumoto, R. R. Synthesis and Pharmacological Characterization of a Novel Sigma Receptor Ligand with Improved Metabolic Stability and Antagonistic Effects Against Methamphetamine. AAPS J. 2012, 14, 43-51, doi:10.1208/s12248-011-9311-8.
40. James, M. L.: Shen, B.: Zavaleta, C.; Nielsen, C. H.: Mesangeau, C.; Vuppala, P. K.: Chan, C.; Avery, B. A.; Fishback, J. A.; Matsumoto, R. R.; et al. New Positron Emission Tomography (PET) Radioligand for Imaging σ-1 Receptors in Living Subjects. J. Med. Chem. 2012, 55, 8272-8282, doi:10.1021/jm300371c.
41. Cunningham, C. E.; Li, S.; Vizeacoumar, F. S.; Bhanumathy, K. K.; Lee, J. S.; Parameswaran, S.: Furber, L.; Abuhussein, O.; Paul, J. M.: McDonald, M.; et al. Therapeutic relevance of the protein phosphatase 2A in cancer. Oncotarget 2016, 7, 61544-61561, doi:10.18632/oncotarget.11399.
42. Morris, G. M.; Huey, R.; Lindstrom, W.; Sanner, M. F.; Belew, R. K.; Goodsell, D. S.; Olson, A. J. AutoDock4 and AutoDockTools4: Automated docking with selective receptor flexibility. J. Comput. Chem. 2009, 30, 2785-2791, doi:10.1002/jcc.21256.
43. Lei, C.; Yang, J.; Hui, J.; Sun, X. On the Calculation of TCID₅₀ for Quantitation of Virus Infectivity. Virol. Sin. 2021, 36, 141-144, doi: 10.1007/s12250-020-00230-5.
44. Winkler, E. S.; Bailey, A. L.; Kafai, N. M.; Nair, S.; McCune, B. T.; Yu, J.; Fox, J. M.; Chen, R. E.; Earnest, J. T.; Keeler, S. P.; Ritter, J. H.; Kang, L.-I.; Dort, S.; Robichaud, A.; Head, R.; Holtzman, M. J.; Diamond, M. S. SARS-CoV-2 infection of human ACE2-transgenic mice causes severe lung inflammation and impaired function. Nature Immunology 2021, 21, 1327-1335. doi: 10.1038/s41590-020-0778-2.

### Embodiments of the Invention

1. A method for treating or preventing SARS-CoV-2 infection in a subject, comprising administration to the subject of a compound, or salt thereof, having sigma receptor binding affinity.
2. A method for treating or preventing symptoms of SARS-CoV-2 infection in a subject, comprising administration to the subject of a compound, or salt thereof, having sigma receptor binding affinity.
3. A method for treating or preventing respiratory symptoms of SARS-CoV-2 infection in a subject, comprising administration to the subject of a compound, or salt thereof, having sigma receptor binding affinity.
4. A method for treating or preventing pulmonary dysfunction associated with SARS-CoV-2 infection in a subject, comprising administration to the subject of a compound, or salt thereof, having sigma receptor binding affinity.
5. A method for treating or preventing cardiovascular dysfunction associated with SARS-CoV-2 infection in a subject, comprising administration to the subject of a compound, or salt thereof, having sigma receptor binding affinity.
6. A method for treating or preventing metabolic dysfunction (e.g., pancreatic function) associated with SARS-CoV-2 infection in a subject, comprising administration to the subject of a compound, or salt thereof, having sigma receptor binding affinity.
7. A method for reducing replication of SARS-CoV-2 virus in a subject, comprising administration to the subject of a compound, or salt thereof, having sigma receptor binding affinity.
8. A method for reducing SARS-CoV-2 virus-induced cellular toxicity in a subject, comprising administration to the subject of a compound, or salt thereof, having sigma receptor binding affinity.
9. The method of any of embodiments 1-8, wherein the compound, or salt thereof, has sigma-1 receptor binding affinity.
10. The method of any of embodiments 1-8, wherein the compound, or salt thereof, has sigma-2 receptor binding affinity.
11. The method of any of embodiments 1-8, wherein the compound, or salt thereof, is a sigma receptor agonist.
12. The method of any of embodiments 1-8, wherein the compound, or salt thereof, is a sigma receptor antagonist.
13. The method of any of embodiments 1-8, wherein the compound, or salt thereof, is AZ66, CM304, CM398, or SA4503, or salt thereof.
14. The method of any of embodiments 1-8, wherein the compound, or salt thereof, is pridopidine, ANAVEX2-73, S1RA, T-817MA, CT1812, roluperidone (MIN-101), or salt thereof.
15. The method of any of embodiments 1-8, wherein the compound, or salt thereof, is diphenhydramine, or salt thereof.
16. The method of any of embodiments 1-15, further comprising administration of an additional therapeutic agent.
17. The method of embodiment 16, wherein the additional therapeutic agent is an antiviral agent, an immunomodulatory agent, an immunosuppressant, an anti-inflammatory agent, an antibody.
18. The method of embodiment 16, wherein the additional therapeutic agent is lactoferrin.
19. A compound that is AZ66, CM304, CM398, or SA4503, or salt thereof, for use in treating or preventing SARS-CoV-2 infection in a subject.
20. A composition comprising a compound that is AZ66, CM304, CM398, or SA4503, or salt thereof, and a pharmaceutically acceptable excipient.
21. The composition of embodiment 20, wherein the composition is for oral, nasal, or injectable administration.

### INCORPORATION BY REFERENCE

All references cited herein are incorporated by reference in their entirety.

### EQUIVALENTS AND SCOPE

In the claims articles such as “a,” “an.” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for treating SARS-CoV-2 infection in a subject, comprising administration to the subject of a compound, or salt thereof, having sigma receptor binding affinity.

2. A method for treating symptoms of SARS-CoV-2 infection in a subject, comprising administration to the subject of a compound, or salt thereof, having sigma receptor binding affinity.

3. The method of claim 2, wherein the symptoms are respiratory symptoms.

4. The method of claim 2, wherein the symptom is pulmonary dysfunction associated with SARS-CoV-2 infection.

5. The method of claim 2, wherein the symptom is cardiovascular dysfunction associated with SARS-CoV-2 infection.

6. The method of claim 2, wherein the symptom is metabolic dysfunction, associated with SARS-CoV-2 infection.

7. A method for reducing replication of SARS-CoV-2 virus or reducing SARS-CoV-2 virus-induced cellular toxicity in a subject, comprising administration to the subject of a compound, or salt thereof, having sigma receptor binding affinity.

8-13. (canceled)

14. The method of claim 1, wherein the compound, or salt thereof, is pridopidine, ANAVEX2-73, S1RA, T-817MA, CT1812, roluperidone (MIN-101), or salt thereof.

15. The method of claim 1, wherein the compound, or salt thereof, is diphenhydramine, or salt thereof.

16. The method of claim 1, further comprising administration of an additional therapeutic agent.

17. (canceled)

18. The method of claim 16, wherein the additional therapeutic agent is lactoferrin.

19. The method of claim 1, wherein the compound is AZ66, CM304, CM398, or SA4503, or salt thereof.

20. A composition comprising a compound that is AZ66, CM304, CM398, or SA4503, or salt thereof, and a pharmaceutically acceptable excipient.

21. (canceled)

22. A method for preventing SARS-CoV-2 infection in a subject, comprising administration to the subject of a compound, or salt thereof, having sigma receptor binding affinity.

23. The method of claim 22, wherein the compound is AZ66, CM304, CM398, or SA4503, or salt thereof.

24. A method for preventing symptoms of SARS-CoV-2 infection in a subject, comprising administration to the subject of a compound, or salt thereof, having sigma receptor binding affinity.

25. The method of claim 24, wherein the symptoms are respiratory symptoms.

26. The method of claim 24, wherein the symptom is pulmonary dysfunction associated with SARS-CoV-2 infection.

27. The method of claim 24, wherein the symptom is cardiovascular dysfunction associated with SARS-CoV-2 infection.

28. The method of claim 24, wherein the symptom is metabolic dysfunction associated with SARS-CoV-2 infection.
